(19) United States
(12) Patent Application Publication
(10) Pub. No.: US 2026/0103773 A1
(43) Pub. Date: Apr. 16, 2026

(54) AGENT FOR SELECTIVE METAL RECOVERY, METAL RECOVERY METHOD, AND METAL ELUTION METHOD

(71) Applicant: Galdieria Co., Ltd., Tokyo (JP)

(72) Inventors: Ayumi Minoda, Tokyo (JP); Xiahui Wang, Tokyo (JP); Mayumi Sato, Tokyo (JP); Rie Yamamoto, Tokyo (JP)

(73) Assignee: Galdieria Co., Ltd., Tokyo (JP)

(21) Appl. No.: 19/350,359

(22) Filed: Oct. 6, 2025

Related U.S. Application Data
(63) Continuation of application No. 16/063,993.

(30) Foreign Application Priority Data
Dec. 22, 2015 (JP) 2015-249567

(51) Int. Cl. C22B 3/18; B01J 20/22; B01J 20/24; B01J 20/34; B01J 45/00; C02F 3/32; C02F 101/20; C12N 1/12; C22B 3/14; C22B 3/16; C22B 3/44; C22B 7/00; C22B 11/00; C22B 59/00

(52) U.S. Cl. CPC C22B 3/18; B01J 20/22; B01J 20/24; B01J 20/345; B01J 45/00; C02F 3/322; C12N 1/12; C22B 3/14; C22B 3/1666; C22B 3/44; C22B 7/00; C22B 11/00; C22B 59/00; B01J 2220/4843; C02F 2101/20; Y02P 10/20

(57) ABSTRACT

The present disclosure provides an agent for selective metal recovery, a metal recovery method, and a metal elution method.

### TECHNICAL FIELD

The present invention relates to an agent for selective metal recovery, a metal recovery method, and a metal elution method.

### BACKGROUND ART

At present, since it is difficult to selectively recover 100 ppm or less of rare earth elements by utilizing a chemical exchange resin or the like, the rare earth elements are discarded as metal effluent. Regarding gold ions as well, several tens ppm or less of gold ions are not recyclable by the current chemical and engineering methods.

Thus, in recent years, metal recovery methods such as a method of eluting metals that are included as solids in a solution by utilizing living organisms (bioleaching), and a method of removing and adsorbing metal ions included in a solution by utilizing living organisms (biosorption), have been developed.

For example, Patent Literature 1 discloses a method of culturing red algae of the order Cyanidiales in a solution and eluting metal ions from metals included as solids in the solution, and a method of causing metal ions in a solution to adsorb to red algae and thereby recovering the metal ions.

Recovery of a metal by a living organism or a biosorbent is useful for metal recovery at low concentrations compared to chemical methods or engineering methods, and such metal recovery has been reported many times as an environment-friendly method enabling reduction of the amount of chemical agents at low cost.

Precious metals such as gold and palladium are indispensable for production of various electronic products. While the amount of electronic products produced continues to increase, these precious metals have limited production. In order to continue producing stable electronic products, it is important to recover precious metals from a metal waste liquid obtained from waste products and recycle the precious metals.

As a method for recovering precious metals, for example, a chemical recovery method using a solvent extraction method or an ion exchange method is known. However, in these methods, it has been difficult to recover precious metals contained in a low concentration (for example, 10 ppm or less) in a metal waste liquid by an economically viable method. In recent years, development of a method that makes it possible to recover precious metals even in low concentrations has proceeded.

As the method that makes it possible to recover precious metals, for example, Patent Literature 2 describes a method for recovering gold and palladium from a metal solution with a hydrochloric acid concentration of 0.1 to 5 M using, as a precious metal adsorbent, a component obtained by treating the green algae belonging to Pseudochoricystis or Chlorella or the residue of the green algae with sulfuric acid.

In addition, for example, Patent Literature 3 proposes a method for recovering metals using live cells of the red algae belonging to the order Cyanidiales. Patent Literature 3 describes that after the acetic acid concentration of a solution containing gold is adjusted to 15 to 400 mM and/or the Cl concentration is adjusted to less than 5 mM, the cells of the red algae are cultured for 24 hours in the solution, whereby gold is recovered by the cells of the red algae.

The disclosure of U.S. Patent Application Publication No. US 2019/0024209 is hereby incorporated by reference for all purposes. The disclosure of U.S. Patent Application Publication No. US 2023/0416866 is also hereby incorporated by reference for all purposes herein.

### CITATION LIST

### Patent Literature

Patent Literature 1: Japanese Unexamined Patent Publication No. 2013-67826.
Patent Literature 2: Japanese Unexamined Patent Publication No. 2012-24752.
Patent Literature 3: Japanese Unexamined Patent Publication No. 2013-67826.

### SUMMARY OF INVENTION

### Problems to be Solved by the Invention

However, recovery of metals by living organisms or biosorbents has a problem that selective recovery or purification of metals is difficult and practicalization has been impeded.

The present invention was achieved in view of the problems described above, and it is an object of the invention to provide an agent for selective metal recovery, a metal recovery method, and a metal elution method, which can be carried out with high efficiency at low cost compared to conventional methods.

However, the method described in Patent Literature 2 has a problem that gold or palladium cannot be satisfactorily recovered from a metal solution having a high hydrochloric acid concentration. For example, in a case where a residue obtained by solvent extraction using an organic solvent is used as a precious metal adsorbent for the microalgae belonging to Pseudochoricystis is used as a precious metal adsorbent, if the hydrochloric acid concentration exceeds 1 M, the adsorption rate of gold and palladium is about 20%. Therefore, the method described in Patent Literature 2 is not a method that can be put into practical use.

The method described in Patent Literature 3 can be used only in a solution or the like in which the acetic acid concentration is adjusted to a low concentration cannot enable gold to be recovered from a metal waste liquid having a high acid concentration, which is actually obtained from waste products. Furthermore, the method proposed in Patent Literature 3 requires management of live cells, adjustment of a solution, and a long recovery time, and thus cannot be said to be a simple and efficient method.

The present invention has been made in view of the above problems. That is, an object of the present invention is to provide a metal recovery agent and a metal recovery method that make it possible to selectively recover precious metals from a solution having a high acid concentration or the like in a simple and efficient manner.

### Means for Solving the Problems

The gist of the present invention is as follows.
[1] An agent for selective metal recovery, the agent including a material derived from an alga belonging to the order Cyanidiales, which is dead cells or a cell surface layer of an alga belonging to the order Cyanidiales, or an artificial material produced by simulating the cell surface layer, or including a porphyrin. Meanwhile, living cells of an alga belonging to the order Cyanidiales may also be used as the material derived from an alga belonging to the order Cyanidiales. Furthermore, it is preferable that the porphyrin is a protonated porphyrin.
[2] The agent for selective metal recovery described in [1] described above, in which the porphyrin is coproporphyrin and/or pheophytin.
[3] The agent for selective metal recovery described in [1] or [2] described above, in which the porphyrin is a protonated compound.
[4] The agent for selective metal recovery described in any one of [1] to [3] described above, in which the agent selectively recovers a noble metal and/or a rare metal including a rare earth element.
[5] The agent for selective metal recovery described in any one of [1] to [4] described above, in which the agent selectively recovers a noble metal including gold or palladium, and/or a lanthanoid from a base metal mixture solution under acidic conditions.
[6] The agent for selective metal recovery described in [5] described above, in which the agent separates and selectively recovers a lanthanoid and iron based on the difference between the ionic radii of the respective elements and the degree of stability of complexes.
[7] The agent for selective metal recovery described in [1] described above, in which the cell surface layer of an alga belonging to the order Cyanidiales adsorbs a noble metal ion complex by an electrostatic interaction or ion exchange and desorbs the noble metal ion complex with a predetermined solution.
[8] The agent for selective metal recovery described in any one of [1] to [7] described above, in which the porphyrin forms nanoparticles by reducing a noble metal.
[9] A metal recovery method, including: an addition step of adding a material derived from an alga belonging to the order Cyanidiales, which is dead cells or a cell surface layer of an alga belonging to the order Cyanidiales, or an artificial material produced by simulating the cell surface layer, or adding a porphyrin, to a metal solution; and a recovery step of recovering a metal from the metal solution by the material derived from an alga belonging to the order Cyanidiales or the porphyrin.
[10] The metal production and recovery method described in [9] described above, in which the porphyrin is coproporphyrin and/or pheophytin.
[11] The metal recovery method described in [9] or [10] described above, in which the recovery step is a step of selectively recovering a noble metal and/or a rare metal including a rare earth element from the metal solution.
[12] The metal recovery method described in any one of [9] to [11] described above, in which the recovery step involves selective recovery of a noble metal including gold or palladium, and/or a lanthanoid from a base metal mixture solution under acidic conditions.
[13] The metal recovery method described in [12] described above, in which the recovery step involves separation and selective recovery of a lanthanoid and iron based on the difference between the ionic radii of the respective elements and the degree of stability of complexes.
[14] The metal recovery method described in any one of [9] to [13] described above, further including a reduction step of forming nanoparticles by causing the porphyrin to reduce a noble metal.
[15] The metal recovery method described in any one of [9] to [14] described above, in which the recovery step involves recovery of gold ions by adsorption using the material derived from an alga belonging to the order Cyanidiales, and the method includes a step of reducing gold ions by a reducing action of the porphyrin.
[16] A metal elution method for eluting a noble metal including gold or palladium, which has been recovered into a material derived from an alga belonging to the order Cyanidiales, which is dead cells or a cell surface layer of an alga belonging to the order Cyanidiales, or an artificial material produced by simulating the cell surface layer, the method including a step of adding a composition for metal elution, which is an acidic solution, to the material derived from an alga belonging to the order Cyanidiales.
[17] A metal elution method for eluting a metal which has been recovered into a material derived from an alga belonging to the order Cyanidiales, which is dead cells or a cell surface layer of an alga belonging to the order Cyanidiales, or an artificial material produced by simulating the cell surface layer, the method including a step of adding a composition for metal elution including a mixed liquid of ammonia and an ammonium salt to the material derived from an alga belonging to the order Cyanidiales.

Furthermore, in regard to the metal recovery method of the present invention, it is preferable that gold ions are recovered and converted to gold nanoparticles by reduction in a short time period, by combining (1) recovery by adsorption utilizing a cell surface layer of an alga belonging to the order Cyanidiales, and (2) reduction of gold ions by a porphyrin, and thereby the purity is increased.

Furthermore, in regard to the metal recovery method of the present invention, it is preferable that a noble metal complex is eluted with a purity of 99.98%, by performing desorption by a particular solution after the (1) recovery by adsorption utilizing a cell surface layer of an alga belonging to the order Cyanidiales.

Furthermore, in regard to the metal recovery method of the present invention, it is preferable that only noble metal ions are purified by burning after the (1) recovery by adsorption utilizing a cell surface layer of an alga belonging to the order Cyanidiales.

In addition, regarding the desorption, a noble metal may be extracted and purified as a complex, by utilizing a mixed liquid of aqueous ammonia and an ammonium salt (ammonium chloride, ammonium sulfate, ammonium carbonate, ammonium bromide, or the like). It is effective for a cell surface layer of an alga belonging to the order Cyanidiales; however, it can also be used with existing ion exchange resins and the like. Meanwhile, it is insufficient with aqueous ammonia only.

That is, regarding the metal recovery method of the present invention, it is preferable to use a mixed liquid of ammonia and an ammonium salt as a solution for eluting a metal which has been recovered into a material derived from an alga belonging to the order Cyanidiales, which is dead cells or a cell surface layer of an alga belonging to the order Cyanidiales, or an artificial material produced by simulating the cell surface layer.

The gist of the present invention is as follows.
[1] A metal recovery agent or a metal compound recovery agent including: a dried matter of a cell of red algae belonging to the order Cyanidiales; a dried matter of a cell derivative of red algae belonging to the order Cyanidiales; or an artificial matter that imitates the dried matter of the cell or the dried matter of the cell derivative.
[2] A method for recovering a metal or a metal compound including: an addition step of adding a dried matter of a cell of the red algae belonging to the order Cyanidiales, a dried matter of a cell derivative of the red algae belonging to the order Cyanidiales, or an artificial matter that imitates the dried matter of the cells or the dried matter of the cell derivative to a metal solution; and an adsorption step of causing a metal or a metal compound contained in the metal solution to be adsorbed onto the cell derived from the dried matter, the cell derivative derived from the dried matter, or the artificial matter.
[3] The method for recovering a metal or a metal compound according to [2], in which an amount of the dried matter or the artificial matter added in the addition step is 0.001 mg or more with respect to 100 ml of the metal solution.
[4] The method for recovering a metal or a metal compound according to [2] or [3], in which the adsorption step is a step of causing at least one metal selected from the group consisting of gold, palladium, ruthenium, platinum, iridium, and osmium, or at least one metal compound containing the metal selected from the group consisting of gold, palladium, ruthenium, platinum, iridium, and osmium contained in the metal solution to be selectively adsorbed.
[5] The method for recovering a metal or a metal compound according to any one of [2] to [4], in which an acid concentration of the metal solution is 0.5 mmol/L or more.
[6] The method for recovering a metal or a metal compound according to [2] or [3], in which the adsorption step is a step of recovering a gold cyanide complex contained in the metal solution.
[7] The method for recovering a metal or a metal compound according to any one of [2] to [6], further including: a step of refining the metal or the metal compound adsorbed onto the cell derived from the dried matter, the cell derivative derived from the dried matter, or the artificial matter.
[8] The method for recovering a metal or a metal compound according to any one of [2] to [6], further including: an elution step of eluting the metal or the metal compound adsorbed onto the cell derived from the dried matter, the cell derivative derived from the dried matter, or the artificial matter using a mixed solution containing ammonia and an ammonium salt.

### Effects of the Invention

According to the present invention, provided is an effect that an agent for selective metal recovery, a metal recovery method, and a metal elution method, by which selective recovery, elution, purification, and the like of a metal can be carried out efficiently at low cost, can be provided.

According to the present invention, it is possible to provide a metal recovery agent and a metal recovery method that make it possible to selectively recover precious metals from a solution having a high acid concentration or the like or a solution containing a cyanide complex in a simple and efficient manner.

### BRIEF DESCRIPTION OF DRAWINGS

FIG. 1A-FIG. 1L are diagrams of graphs showing the ICP-MS measurement results of the concentrations of various metals included in a culture fluid supernatant and cell fractions at the time of non-addition/addition of cells.

FIG. 2 is a diagram (photograph as a substitute for a diagram) showing a visible light image and a UV irradiation image of a fraction including ethyl acetate in a medium supernatant (ethyl acetate-extracted fraction obtained by mixing a medium supernatant and ethyl acetate).

FIG. 3 is a diagram of a graph showing the absorption wavelength on the horizontal axis and the light absorbance on the vertical axis for a fraction including ethyl acetate in a medium supernatant (ethyl acetate-extracted fraction obtained by mixing a medium supernatant and ethyl acetate).

FIG. 4 is a diagram showing an ultraviolet-visible light absorption spectrum obtained before HPLC purification.

FIG. 5 is a diagram showing an ultraviolet-visible light absorption spectrum obtained after HPLC purification.

FIG. 6 is a diagram showing the MS/MS analysis results for a colorant purified by HPLC.

FIG. 7 is a diagram showing the 1H-NMR analysis results for a colorant purified by HPLC.

FIG. 8 is a diagram showing the spectra obtained at the time of no metal addition (HPLC purification product (coproporphyrin) of an ethyl acetate-extracted fraction: dotted lines in the diagram), at the time of metal addition of Nd³⁺, Dy³⁺, Fe²⁺, and Fe³⁺ (HPLC purification product (coproporphyrin) of an ethyl acetate-extracted fraction+metals: solid lines in the diagram), and at the time of EDTA addition (HPLC purification product (coproporphyrin) of an ethyl acetate-extracted fraction+metals+EDTA: broken lines in the diagram).

FIG. 9A-FIG. 9C are diagrams of graphs showing the recovery efficiencies for gold, platinum, and palladium achieved by G. sulphuraria.

FIG. 10 is a diagram of a graph showing the recovery efficiencies of living cells (Living Cells) and dead cells (Freeze-thawed Cells) of G. sulphuraria.

FIG. 11 is a diagram (photograph as a substitute of a diagram) showing the gold ion concentrations added to cells and the color changes of culture fluids.

FIG. 12 is a diagram (photographs as substitutes of diagrams) showing the positions of gold nanoparticles in a microscopic image of cells and the results for an Au composition analysis by TEM-EDS.

FIG. 13 is a diagram of a graph showing the recovery rates at gold ion concentrations of 0.5 to 25.0 ppm and incubation times.

FIG. 14 is a diagram of a graph showing the pH-dependent recovery rates for gold ions into cells.

FIG. 15 is a diagram (photograph as a substitute for a diagram) showing color changes of culture fluids depending on pH and the incubation time.

FIG. 16 is a diagram (photograph as a substitute for a diagram) showing color changes of culture fluids in the case of performing incubation at various temperatures in a dark place and a bright place.

FIG. 17 is a flow chart showing a method for producing various cell fractions.

FIG. 18 is a diagram (photograph as a substitute for a diagram) showing color changes obtained after adding gold ions to various cell fractions and culturing the cell fractions for 30 minutes.

FIG. 19 is a diagram (photograph as a substitute for a diagram) showing the results obtained by incubating a methanol-extracted fraction by varying the pH.

FIG. 20 is a diagram (photograph as a substitute for a diagram) showing the results obtained by incubating a methanol-extracted fraction by varying the gold ion concentration.

FIG. 21 is a diagram (photographs as a substitute for a diagram) showing CCD camera images and SEM images of a MeOH-extracted fraction and gold-colored structures produced by incubation of gold ions at a high concentration.

FIG. 22 is a diagram of a graph showing spectral shifts at the visible light portion obtained in a case in which gold ions were added to coproporphyrin.

FIG. 23 is a diagram (photographs as a substitute for a diagram) showing the results obtained by adding gold ions to preparations of coproporphyrin and pheophytin and incubating the preparations overnight.

FIG. 24 is a diagram schematically illustrating a first stage of biosorption (adsorption) and a second stage of reduction.

### EMBODIMENTS FOR CARRYING OUT THE INVENTION

Hereinafter, the agent for selective metal recovery, metal recovery method, and metal elution method of the present invention will be described in detail. First, the present embodiments will be described together with the background from which the present invention was devised, and subsequently, Examples accompanied by experimental results will be described. In addition, this invention is not intended to be limited by the following present embodiments and Examples. For example, in the following embodiments and Examples, algal bodies of red algae belonging to the order Cyanidiales, or a cell surface layer, an algal body adsorbent, and cell surface layer fractions of red algae may be described; however, the present invention is not intended to be limited to these, and the present invention may also be applied to materials derived from algae belonging to the order Cyanidiales, such as dead cells and a cell surface layer of an alga belonging to the order Cyanidiales, and an artificial material produced by simulating the cell surface layer.

### Embodiments

An overview of the agent for selective metal recovery, the metal recovery method, and the metal elution method according to the present embodiments will be described. As described above, recovery of a metal by a living organism or a biosorbent is a method that is useful for recovery of a metal at a low concentration or imposes less environmental burden by enabling reduction of the amount of chemical agents at low cost compared to a chemical method or an engineering method; however, selective recovery or purification of metals is difficult, and practicalization has been impeded. Therefore, recovery of several tens ppm or less of noble metal ions by any one of a chemical method, an engineering method, and a biological method is difficult, and noble metal ions are discarded as metal effluent.

Thus, the inventor of the present invention conducted a thorough investigation, and as a result, the inventor found that red alga of Galdieria sulphuraria (hereinafter, referred to as “G. sulphuraria”) belonging to the order Cyanidiales performs elution or recovery into cells of rare earth elements from neodymium magnet waste materials depending on the culture conditions. Furthermore, the inventor of the present invention discovered that selectivity between rare earth elements and iron is exhibited in the elution or recovery thereof (see Example 1 described below).

The incubation time after cells of G. sulphuraria are added to a metal solution containing neodymium magnet waste materials and the like is not particularly limited; however, the incubation time is preferably 1 minute to 24 hours, and more preferably 10 minutes to 30 minutes. Furthermore, the incubation temperature is not particularly limited; however, the incubation temperature is preferably 0° C. to 70° C. When incubation is performed under the above-described conditions of incubation time and/or incubation temperature, the efficiency for elution or recovery into cells of rare earth elements tends to increase.

Next, the inventor of the present invention identified coproporphyrin as a chelator in relation to the elution of rare earth elements (see Example 2 described below). Furthermore, the inventor of the present invention found that coproporphyrin chelates rare earth elements or divalent iron, while not chelating trivalent iron.

Since iron exists in a trivalent form under acidic conditions, the inventor of the present invention found that only those rare earth elements can be selectively chelated, even in the presence of iron, using protonated coproporphyrin by leaving the compound under acidic conditions. That is, the inventor discovered that this is an important mechanism by which selectivity between rare earth elements and iron in red algae such as G. sulphuraria is exhibited.

That is, according to an embodiment of the present invention based on this discovery, a noble metal such as gold or palladium, or a rare earth element such as a lanthanoid is selectively recovered from a base metal mixture solution of iron and the like, by using a porphyrin such as coproporphyrin and leaving the compound under acidic conditions. As such, when a porphyrin such as coproporphyrin is utilized under acidic conditions, even in a case in which base metals such as iron exist in large quantities, noble metals or rare earth elements can be selectively recovered from metal effluent. Furthermore, regarding the reason why trivalent iron is not chelated but trivalent rare earth elements are chelated, the differences in the ionic radius and the degree of stability of complexes may be considered as the causative factors. Therefore, even between rare earth elements that have very similar properties and are currently not easily separable industrially (for example, Dy and Tb), separation can be achieved based on the difference in the ionic radius or the difference in the degree of stability of complexes.

Furthermore, the inventor of the present invention found that in the phenomenon in which red alga G. sulphuraria reduces gold ions in the presence of light and thereby forms nanoparticles (Example 7 described below), a porphyrin such as coproporphyrin or pheophytin accelerates reduction of gold. Here, the inventor found that coproporphyrin forms gold particles of larger sizes, compared to pheophytin, which is a kind of the same porphyrin (Example 8 described below).

That is, according to an embodiment of the present invention based on this discovery, gold particles are formed by reducing gold ions in a solution by using a porphyrin such as coproporphyrin or pheophytin. In addition, based on the same principle, according to an embodiment of the present invention, a solid metal may also be formed by using a porphyrin and reducing a metal having a high oxidation-reduction potential, such as a noble metal ion, in a solution.

As described above, as a result of thorough investigation of the inventor of the present invention, the inventor finally devised an invention, by which: (1) a porphyrin works as a chelator to selectively adsorb (chelate) metal ions of a noble metal or a rare earth element; (2) a metal complex of a noble metal, a rare earth element, or the like can be selectively adsorbed from a base metal mixture solution of iron and the like by leaving the solution on a cell surface layer of an alga belonging to the order Cyanidiales under acidic conditions; and (3) noble metal ions are reduced and converted to solid particles by using a porphyrin.

A porphyrin is a compound existing in all living organisms including from microorganisms to human beings, and in recent years, chemical synthesis methods have also been developed. By utilizing a porphyrin derived from a living organism or chemical synthesis, an inexpensive, highly efficient method compared to conventional methods can be provided in connection with selective recovery of rare earth elements at low concentrations, which are currently not recycled, or nanoparticle formation or recovery based on reduction of gold ions.

### [Embodiments of Composition for Metal Elution/Metal Elution Method]

An overview of the composition for metal elution/metal elution method of the present embodiments will be described. As described above, recovery of a metal by a living organism or a biosorbent is a method that is useful for recovery of a metal at a low concentration or imposes less environmental burden by enabling reduction of the amount of chemical agents at low cost compared to a chemical method or an engineering method; however, selective recovery or purification of metals is difficult, and practicalization has been impeded. Therefore, recovery of several tens ppm or less of noble metal ions by any one of a chemical method, an engineering method, and a biological method is difficult, and noble metal ions are discarded as metal effluent.

Conventionally, as a method for purifying a noble metal collected by an alga or a microorganism, methods of using elution by a mixed solution of thiourea (thiourea) and hydrochloric acid, which is utilized for leaching from minerals, or using combustion have been disclosed. Since elution methods using thiourea are disadvantageous from the viewpoints of economic efficiency and environment, and also it is difficult to apply the elution methods to subsequent chemical processes, practicalization has not been achieved. Furthermore, also for combustion methods, being disadvantageous from the viewpoints of economic efficiency and environment has been a problem.

The inventor of the present invention conducted a thorough investigation, and as a result, the inventor discovered that algae belonging to the order Cyanidiales recover noble metals at low concentrations with high efficiency (see Examples 3 and 4 described below). Base on this discovery, the inventor of the present invention has devised a method of selectively recovering noble metals into algal cells by utilizing an alga belonging to the order Cyanidiales by adjusting the acid concentration of a metal effluent (aqua regia solution) including gold and palladium to about 0.5 M (see Example 5 described below), and extracting and purifying only noble metals from a material derived from an alga belonging to the order Cyanidiales, which is dead cells or a cell surface layer of an alga belonging to the order Cyanidiales, or an artificial material produced by simulating the cell surface layer (see Example 6 described below).

An embodiment of the present invention based on these findings is to provide a composition for metal elution, which is an acidic solution, for eluting a noble metal such as gold or palladium which has been recovered into a material derived from an alga belonging to the order Cyanidiales, such as an algal body adsorbent such as an algal body of a red alga or a cell surface layer of a red alga. In other words, an embodiment of the present invention relates to a metal elution method of eluting a noble metal such as gold or palladium which has been recovered into a material derived from an alga belonging to the order Cyanidiales, the method including a step of adding a composition for metal elution, which is an acidic solution, to an algal body of a red alga or an algal body adsorbent. The composition for metal elution is not particularly limited; however, from the viewpoint of increasing the efficiency of elution, it is preferable to use an acidic solution including aqua regia. Furthermore, the acid concentration of the acidic solution is not particularly limited; however, from the viewpoint of increasing the efficiency of elution, the acid concentration is preferably 0.1 M to 10 M, more preferably 0.1 M to 1.0 M, and particularly preferably 0.3 M to 0.8 M. Furthermore, an embodiment of the present invention includes a step of adding a composition for metal elution including a mixed liquid of ammonia and an ammonium salt, which is intended for eluting a metal which has been recovered into a material derived from an alga belonging to the order Cyanidiales, to an algal body of a red alga or an algal body adsorbent (see Example 6 described below).

Thereby, the noble metal ions adsorbed to the material derived from an alga belonging to the order Cyanidiales, including an algal body of a red alga, dead cells of a red alga, and the like, can be eluted with high purity, and methods such as bioleaching and biosorption can be further improved. Therefore, the present invention can contribute to recovery and purification of noble metals with high efficiency at low cost compared to conventional methods, by using red algae.

Hereinafter, embodiments of the present invention will be described, but the present invention is not limited to the following embodiment without departing from the spirit of the present invention.

[Metal or Metal Compound Recovery Agent] A metal or a metal compound recovery agent of the present invention includes the dried matter of cells of the red algae belonging to the order Cyanidiales (hereinafter, also simply referred to as “cell”), the dried matter of cell derivatives of the red algae belonging to the order Cyanidiales, or an artificial matter that imitates the dried matter of cells or the dried matter of cell derivatives (hereinafter, also simply referred to as “artificial matter”). Here, examples of the “artificial matter produced by imitating the dried matter” include those produced by organic synthesis or the like.

The red algae belonging to the order Cyanidiales are not particularly limited, and for example, red algae belonging to the genus Galdieria, Cyanidium, or Cyanidioshyzon can be used. Among these red algae, those belonging to the genus Galdieria are preferable, and Galdieria sulphuraria (hereinafter, referred to as “G. sulphuraria”) is more preferable. It has been confirmed by the present inventor's research (Japanese Patent Application No. 2015-249567) that the red algae belonging to the order Cyanidiales selectively recover precious metals from metal solutions with the inside of the cells or the surface layers of the cells. Therefore, the “cell derivatives” of the red algae belonging to the order Cyanidiales may be “cell inside derivatives” or “cell surface layer derivatives”.

The dried matter used in the present invention can be obtained by performing a drying treatment on the cells or cell derivatives of the red algae belonging to the order Cyanidiales. The method of the drying treatment is not particularly limited, and for example, known methods such as a spray drying treatment, a freeze-drying treatment, and a decompression drying treatment can be used. By subjecting the cells to the drying treatment, the acid resistance of the cells is improved, and precious metals can be recovered even from a metal waste liquid having a high acid concentration without the need for dilution or the like. In addition, by subjecting the cells to the drying treatment, it becomes possible to improve the handleability compared to live cells.

The culture conditions of the cells to be subjected to the drying treatment are not particularly limited and for example, may be any conditions including photoautotrophic conditions for growth only by photosynthesis, photomixotrophic conditions for growth by both photosynthesis and the metabolism of organic matter, heterotrophic conditions for growth only by the metabolism of organic matter in the dark, quasi-anaerobic autotrophic conditions for growth by photosynthesis under quasi-anaerobic conditions, and quasi-anaerobic heterotrophic conditions for fermentation in the dark under quasi-anaerobic conditions.

Further, the period at which the cells to be subjected to the drying treatment are harvested is not particularly limited, and may be any of the lag phase, exponential phase, stationary phase, and death phase. The cells to be subjected to the drying treatment are not limited to live cells, and may be dead cells. Among these, from the viewpoint of improving the metal recovery efficiency, cells in the exponential phase, stationary phase, or death phase are preferable.

[Method of Recovering Metal or Metal Compound] A method for recovering a metal or a metal compound of the present invention includes: an addition step of adding the dried matter of cells of the red algae belonging to the order Cyanidiales, the dried matter of cell derivatives of the red algae belonging to the order Cyanidiales, or an artificial matter that imitates the dried matter of cells or the dried matter of cell derivatives to a metal solution; and an adsorption step of causing a metal or a metal compound contained in the metal solution to be adsorbed onto the cells derived from the dried matter, the cell derivatives derived from the dried matter, or the artificial matter.

Examples of metals that can be selectively recovered by the present invention include precious metals such as gold, palladium, platinum, ruthenium, rhodium, osmium, and iridium and the like. Among these, it is possible to recover gold, palladium, platinum, ruthenium, osmium, and iridium with high efficiency.

The precious metals may be contained in the metal solution as a solid of a simple metal or a metal salt, an ion, or a complex. Even in a case where the precious metals are contained in the metal solution as a solid, the solid can be eluted (bioleached) in the metal solution as a metal ion by the cells or the cell derivatives derived from the dried matter or the artificial matter so as to be recovered.

The metal solution is not particularly limited as long as it contains one or more of the above-mentioned precious metals, and may also contain a base metal such as copper or iron in addition to the above-mentioned precious metals. According to the present invention, it is possible to selectively recover a precious metal from a solution in which the precious metal and a base metal are mixed.

The metal solution is not particularly limited as long as it can dissolve the above-mentioned precious metals or the like, and is preferably, for example, a solution in which the above-mentioned precious metals can be present as a complex having a negative charge. The complex having a negative charge is not particularly limited, is preferably, for example, a chloride complex, a hydroxide complex, or a cyanide complex.

Specific examples of the metal solution in which the above-mentioned precious metal can be present as a complex having a negative charge include an acid solution containing, for example, hydrochloric acid or aqua regia, or a solution containing a cyanide complex. These solutions are, for example, solutions used to dissolve precious metals contained in waste products, that is, solvents for metal waste liquids that are industrially discharged. According to the present invention, it is possible to selectively recover precious metals from these metal waste liquids. In addition, the metal solution of the present invention is not necessarily limited to the metal waste liquids that are industrially discharged.

The acid concentration in a case where the metal solution contains an acid solution is not particularly limited, and can be appropriately adjusted according to the kind of acid solution and the kind or concentration of the precious metal as a recovery object. For example, the acid concentration is preferably 0.5 M (mol/L) or more, more preferably 1 M or more, and even more preferably 2 M or more. Moreover, the acid concentration is, for example, preferably 13 M or less, and more preferably 6 M or less. In a case where the acid concentration is less than 0.5 M, there is concern that the metal may not be sufficiently dissolved depending on the kind of the metal. In a case where the acid concentration exceeds 13 M, there is concern that the metal recovery efficiency may decrease. However, but this can be compensated by increasing the addition amount or reaction time.

Hereinafter, a preferable acid concentration for each kind of metal to be a recovery object. The acid concentration in a case where the recovery object is gold is, for example, preferably 0.5 mM or more, more preferably 5 mM or more, and even more preferably 500 mM or more. In addition, the acid concentration in the case where the recovery object is gold is, for example, preferably 13 M or less, more preferably 10 M or less, and even more preferably 6 M or less.

The acid concentration in a case where the recovery object is palladium is, for example, preferably 0.5 mM or more, more preferably 5 mM or more, and even more preferably 500 mM or more. In addition, the acid concentration in the case where the recovery object is palladium is, for example, preferably 13 M or less, more preferably 10 M or less, and even more preferably 6 M or less.

The acid concentration in a case where the recovery object is platinum is, for example, preferably 5 mM or more, more preferably 50 mM or more, and even more preferably 0.1 M or more. In addition, the acid concentration in the case where the recovery object is platinum is, for example, preferably 10 M or less, more preferably 4 M or less, and even more preferably 2 M or less.

The acid concentration in a case where the recovery object is ruthenium is, for example, preferably 5 mM or more, more preferably 50 mM or more, and even more preferably 0.1 M or more. In addition, the acid concentration in the case where the recovery object is ruthenium is, for example, preferably 10 M or less, more preferably 4 M or less, and even more preferably 2 M or less.

The acid concentration in a case where the recovery object is rhodium is, for example, preferably 5 mM or more, more preferably 50 mM or more, and even more preferably 0.1 M or more. In addition, the acid concentration in the case where the recovery object is rhodium is, for example, preferably 10 M or less, more preferably 4 M or less, and even more preferably 1 M or less.

The acid concentration in a case where the recovery object is osmium is, for example, preferably 5 mM or more, more preferably 50 mM or more, and even more preferably 0.1 M or more. In addition, the acid concentration in the case where the recovery object is osmium is, for example, preferably 10 M or less, more preferably 4 M or less, and even more preferably 1 M or less.

The acid concentration in a case where the recovery object is iridium is, for example, preferably 5 mM or more, more preferably 50 mM or more, and even more preferably 0.1 M or more. In addition, the acid concentration in the case where the recovery object is iridium is, for example, preferably 10 M or less, more preferably 4 M or less, and even more preferably 1 M or less.

According to the present invention, since precious metals can be recovered even from a metal solution having a high acid concentration as described above, an operation of excessively diluting an actual metal waste liquid or the like is not required, and excellent efficiency is achieved. Furthermore, a problem of an excessive increase in the volume of the metal waste liquid by dilution can also be prevented, so that excellent space saving is achieved.

The amount of the dried matter added in the addition step is not particularly limited, and can be appropriately adjusted according to the amount or acid concentration of the metal solution or the kind or concentration of the precious metal as a recovery object, but is, for example, preferably 0.001 mg or more, more preferably 0.01 mg or more, and even more preferably 0.1 mg or more with respect to 100 ml of a metal solution in which the concentration of the metal as a recovery object is 1 ppm. In addition, the amount of the dried matter added in the addition step is, for example, preferably 1000 g or less, more preferably 100 g or less, and even more preferably 10 g or less with respect to 100 ml of a metal solution in which the concentration of the metal as a recovery object is 1 ppm. In a case where the amount of the dried matter added is less than 0.001 mg with respect to 100 ml of the metal solution in which the concentration of the metal as a recovery object is 1 ppm, the metal recovery efficiency tends to decrease. In addition, even if the amount of the dried matter added exceeds 1000 g with respect to 100 ml of the metal solution in which the concentration of the metal as a recovery object is 1 ppm, no difference in the metal recovery efficiency tends to be observed. It is preferable to increase the amount of the added dried matter as the metal concentration or the acid concentration increases. A decrease in the metal recovery rate due to a decrease in the addition amount can be compensated by adjusting the acid concentration or increasing the reaction time.

The pH in a case where the metal solution is a solution containing a cyanide complex is not particularly limited, and can be appropriately adjusted according to the kind of solution and the kind or concentration of the precious metal as a recovery object, but is, for example, preferably a pH of 2 or more, more preferably a pH of 4 or more, and even more preferably a pH of 10 or more. The pH of the solution containing a cyanide complex is, for example, preferably a pH of 14 or less. In a case where the pH of the solution containing a cyanide complex is less than 2, the metal recovery efficiency tends to decrease. Even if the pH of the solution containing a cyanide complex exceeds 14, the metal recovery efficiency tends to decrease.

The adsorption step can be performed, for example, by stirring the metal solution, to which the dried matter of cells is added, for a predetermined time. The time for performing the adsorption step is not particularly limited, and can be appropriately adjusted according to the kind of solution and the kind or concentration of the precious metal as a recovery object, but is, for example, preferably 1 minute or longer, more preferably 3 minutes or longer, and even more preferably 15 minutes or longer. In addition, the time for performing the adsorption step is, for example, preferably 24 hours or shorter. In a case where the time for performing the adsorption step is less than one minute, there is concern that the metal may not be recovered sufficiently. Even if the time for performing the adsorption step exceeds 24 hours, no difference in the metal recovery efficiency tends to be observed.

The temperature at which the adsorption step is performed is not particularly limited, and can be appropriately adjusted according to the kind of solution and the kind or concentration of the precious metal as a recovery object, but is for example, preferably 0° C. or higher and more preferably 4° C. or higher. In addition, the temperature at which the adsorption step is performed is, for example, preferably 100° C. or lower, more preferably 80° C. or lower, and even more preferably 60° C. or lower. In a case where the temperature at which the adsorption step is performed is lower than 0° C., there is concern that the metal recovery efficiency may decrease. Even in a case where the temperature at which the adsorption step is performed exceeds 100° C., there is concern that the metal recovery efficiency may decrease.

The method for recovering a metal or a metal compound of the present invention preferably further includes, after the adsorption step, a step of refining the metal or the metal compound adsorbed onto the cells or the like. The step of refining the metal and the like is not particularly limited, and for example, a method of burning the cells and the like, or a method of dissolving the cells or the surface layer of the cells (for example, a residue bonded to the precious metal complex) to elute the metal can be applied. Specifically, for example, a method of eluting a metal or the like using a predetermined metal elution solution such as an acidic thiourea solution, a mixed solution containing ammonia and an ammonium salt, an acid solution (for example, a hydrochloric acid solution or aqua regia), an alkaline solution (for example, KOH solution), or a metal chelate solution (for example, EDTA solution) can be applied and implemented. Among these methods, from the viewpoint of enhancing the refinement efficiency, a method of eluting a metal and the like using, as the metal elution solution, an acidic thiourea solution or a mixed solution containing ammonia and an ammonium salt is preferable. In addition, from the viewpoint of increasing the refinement efficiency while reducing the cost and the burden on the environment, a method of eluting a metal and the like using, as the metal elution solution, a mixed solution containing ammonia and an ammonium salt is preferable.

Therefore, the method for recovering a metal or a metal compound of the present invention preferably further includes an elution step of eluting the metal or the metal compound adsorbed onto the cells derived from the dried matter or the cell derivatives derived from the dried matter using the mixed solution containing ammonia and an ammonium salt. The metal adsorbed onto the cells and the like in the adsorption step can be eluted using the mixed solution containing ammonia and an ammonium salt. The ammonium salt used for the mixed solution is not particularly limited, and for example, ammonium chloride, ammonium sulfate, ammonium carbonate, and ammonium bromide can be used. Elution cannot be achieved only by an ammonia solution or a solution of an ammonium salt (Ju et al., Bioresource Technology 211(2016), 754-766).

The concentration of the ammonium salt in the mixed solution is not particularly limited, but is, for example, preferably 0.02 M or more, more preferably 0.1 M or more, and even more preferably 0.2 M or more. The concentration of the ammonium salt in the mixed solution is, for example, preferably 1 M or less, more preferably 0.6 M or less, and even more preferably 0.4 M or less. In a case where the concentration of the ammonium salt is less than 0.02 M, the elution rate of the metal adsorbed onto the cells tends to decrease. Also in a case where the concentration of the ammonium salt exceeds 1 M, the elution rate of the metal adsorbed onto the cells tends to decrease.

The pH of the mixed solution is not particularly limited, but is, for example, preferably a pH of 3 or more, more preferably a pH of 7 or more, and even more preferably a pH of 11 or more. The pH of the mixed solution is, for example, preferably a pH of 14 or less, more preferably a pH of 13 or less, and even more preferably a pH of 12 or less. In a case where the pH of the mixed solution is less than 3, the elution rate of the metal adsorbed onto the cells tends to decrease.

### EXAMPLES

Subsequently, in order to demonstrate the agent for selective metal recovery, the composition for metal elution, the metal production method, and the metal elution method according to the embodiments of the present invention, Example 1 to Example 9 carried out by the inventors of the present invention will be described.

### Example 1

Example 1 relating to the elution (bioleaching) and recovery into cells (biosorption) of rare earth elements from a neodymium magnet waste material will be described.

### <Method>

First, a neodymium magnet waste material containing iron as a main component [4.7 g of Fe²⁺/³⁺, 1.7 g of Nd³⁺, 0.5 g of praseodymium (Pr³⁺), and 0.4 g of Dy³⁺ (see the following table) in 10 g] was added to 20 ml of a G. sulphuraria culture fluid. More specifically, regarding the culture conditions, the cell density in 10 mg of neodymium magnet waste material/20 ml of 2×Allen's medium was adjusted to 10⁸ cells/ml.

TABLE 1

| | | |
|---|---|---|
| Fe | 4.7 | [235 ppm*] |
| Nd | 1.7 | [85 ppm*] |
| Pr | 0.5 | [25 ppm*] |
| Dy | 0.4 | [20 ppm*] |
| Others | | |
| (Tb, B, Co, C, Al) | 0.2 | |
| H₂O | 2.5 | |
| Total | 10 g | |

*Concentrations in the case of being totally dissolved in 20 ml

Subsequently, red alga G. sulphuraria was cultured for five days under the following five different culture conditions.
(1) Photoautotrophic conditions (Light) in which cells proliferate only by photosynthesis.
(2) Photomixotrophic conditions (Light+Glc) in which both photosynthesis and metabolism of organic materials are carried out.
(3) Heterotrophic conditions (Dark+Glc) in which only organic materials are metabolized in the dark.
(4) Semianaerobic autotrophic conditions (Light) in which cells proliferate by photosynthesis under semianaerobic conditions attained by implementing forced ventilation with 100% carbon dioxide.
(5) Semianaerobic heterotrophic conditions (Dark+Acetate) in which fermentation is carried out in the dark under semianaerobic conditions attained by implementing forced ventilation with 100% nitrogen.

Then, the concentrations of the various metals included in the culture fluid supernatants and cell fractions were determined by ICP-MS on Day 0, Day 2, and Day 5 of culture.

### <Results>

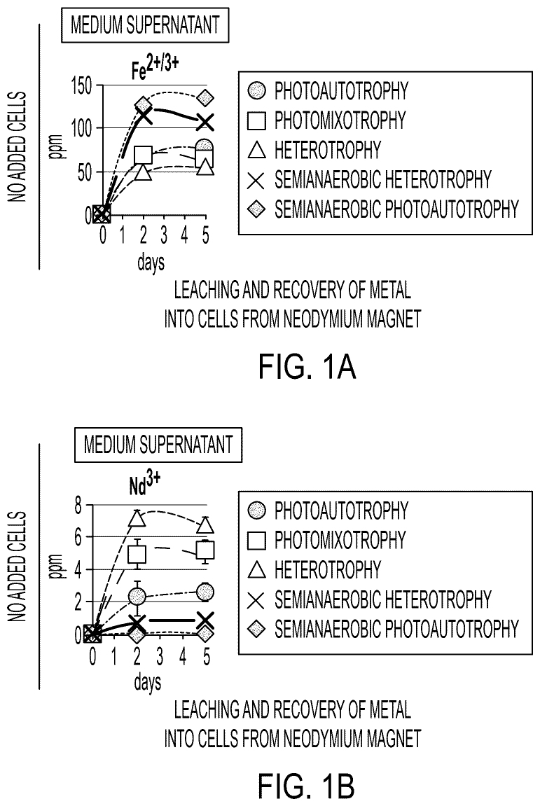

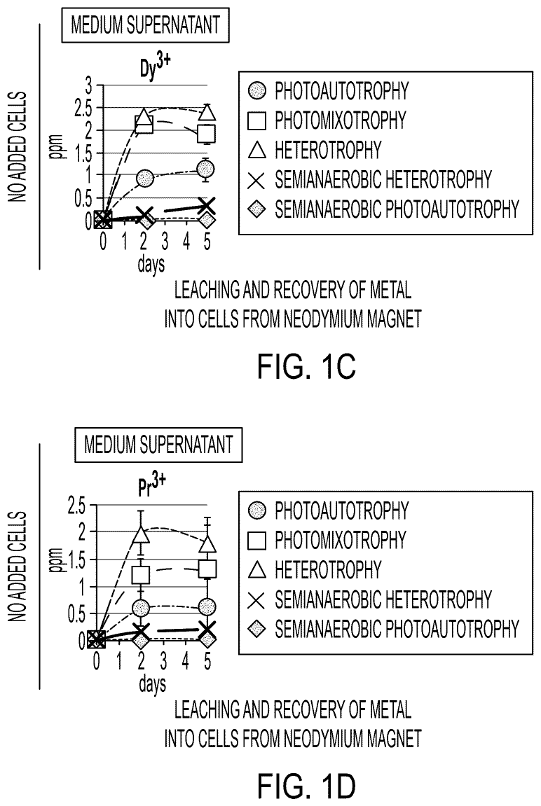

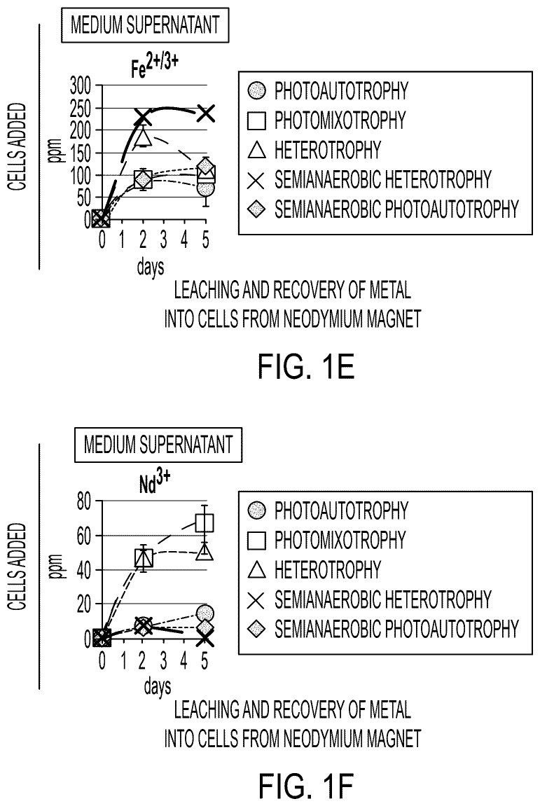

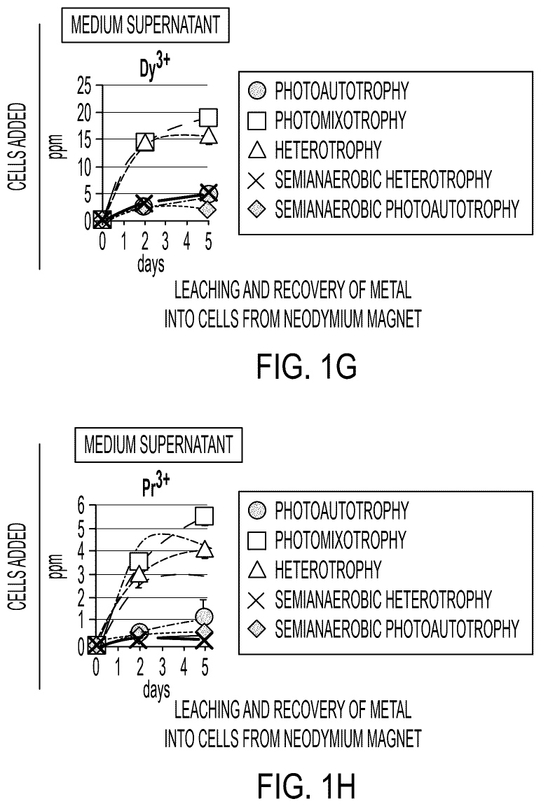

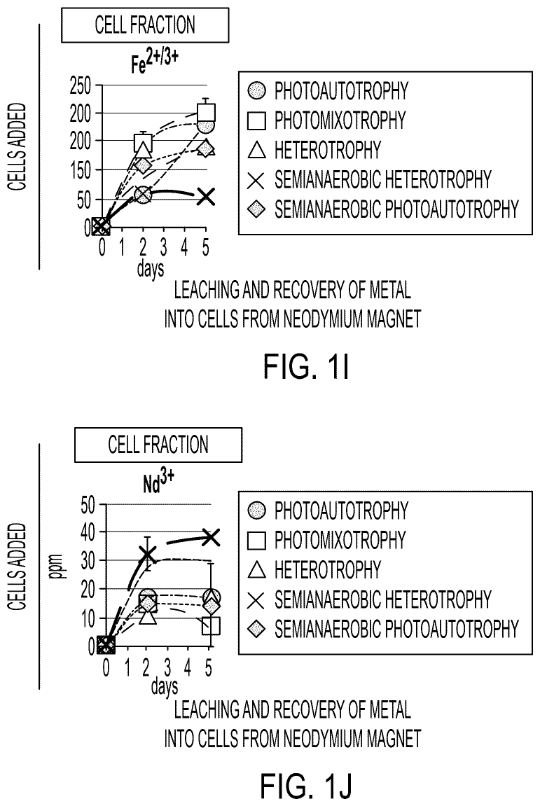

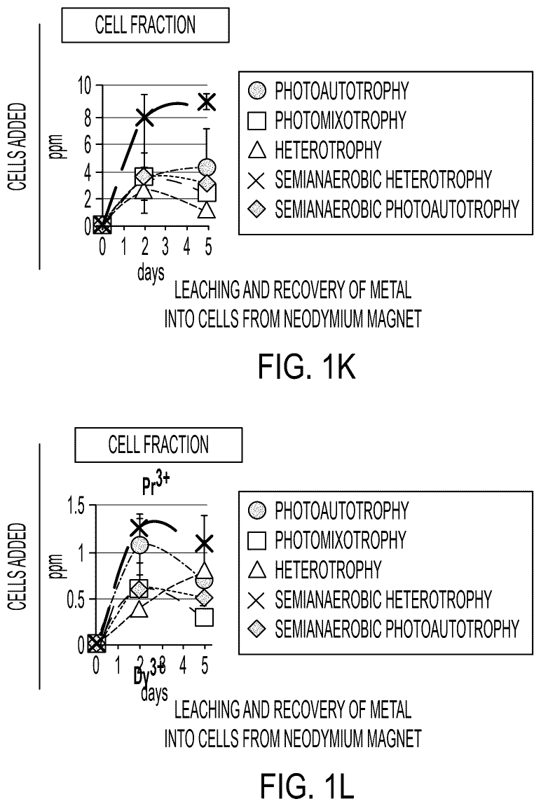

FIG. 1A-FIG. 1L are diagrams of graphs showing the ICP-MS results for the concentrations of the various metals included in the culture fluid supernatants and cell fractions at the time of non-addition/addition of cells. As shown in FIG. 1A-FIG. 1L, in a case in which only a neodymium magnet waste material was added to the culture fluid, and cells were not added, iron was eluted into the culture fluid; however, rare earth elements (Nd³⁺, Dy³⁺, and Pr²⁺) hardly dissolved in the medium (FIG. 1A-FIG. 1L).

In contrast, in a case in which G. sulphuraria cells were added to the culture fluid together with a neodymium magnet waste material, the concentrations of the rare earth elements in the culture fluid supernatants increased under the (2) photomixotrophic conditions and the (3) heterotrophic conditions (FIG. 1F-FIG. 1H). The concentrations of iron in the culture fluid supernatants under the (2) photomixotrophic conditions and the (3) heterotrophic conditions were not different from the concentrations obtained without addition of cells, and the conditions in which the concentration of iron in the culture fluid supernatant was the highest were the (5) semianaerobic heterotrophic conditions (FIG. 1E).

Next, when the metal concentrations in the cell fractions in a case in which a neodymium magnet waste material and cells were added to the culture fluid were examined, the concentrations of the rare earth elements were the highest under the (5) semianaerobic heterotrophic conditions (FIG. 1J-FIG. 1L). In contrast, the concentration of iron in the cell fraction under these (5) semianaerobic heterotrophic conditions was the lowest among the five culture conditions (FIG. 1L).

### SUMMARY

<1> It was found that when cells of G. sulphuraria are added to the medium, elution of iron and rare earth elements occurs more efficiently in the culture fluid supernatant. <2> It was found that the concentrations of iron and rare earth elements in the culture fluid supernatant or the cell fraction vary depending on the culture conditions for G. sulphuraria. <3> It was found that not only the elution of rare earth elements from a neodymium magnet waste material into the medium supernatant but also concentration of rare earth elements into the cell fraction occur under semianaerobic conditions.

### DISCUSSION

Conventionally, in a bioleaching process utilizing microorganisms, a step of recovering metals from the solution after a step of eluting metals from a metal waste material or mineral ore (bioleaching) is needed. However, findings were obtained that when G. sulphuraria is utilized, not only rare earth elements can be eluted into a medium-dissolved culture fluid supernatant, but also the rare earth elements can be recovered into cells, and thus, two steps of elution and recovery in conventional cases can be combined into one step.

### Example 2

Example 2 relating to the identification of a chelator exhibiting selectivity for rare earth elements will be described below.

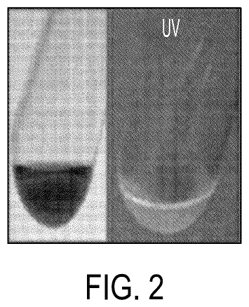

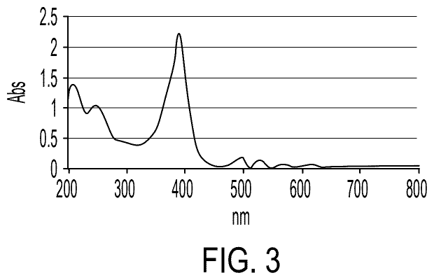

From the results of Example 1, it was predicted that the medium supernatant obtained under the (2) photomixotrophic conditions includes a chelator exhibiting high affinity for rare earth elements, compared to iron. Thus, a fraction including ethyl acetate was fractionated from the medium supernatant, and the optical characteristics were investigated. FIG. 2 is a diagram (photograph as a substitute for a diagram) showing a visible light image and a UV irradiation image of a fraction including ethyl acetate in a medium supernatant (ethyl acetate-extracted fraction obtained by mixing a medium supernatant and ethyl acetate). FIG. 3 is a diagram of graphs showing the absorption wavelength on the horizontal axis and the light absorbance on the vertical axis for a fraction including ethyl acetate in a medium supernatant (ethyl acetate-extracted fraction obtained by mixing a medium supernatant and ethyl acetate).

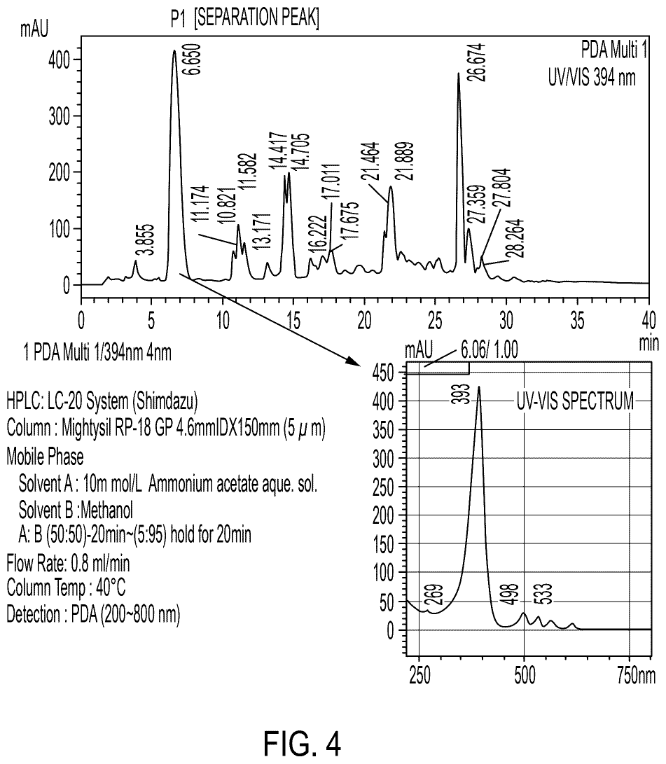

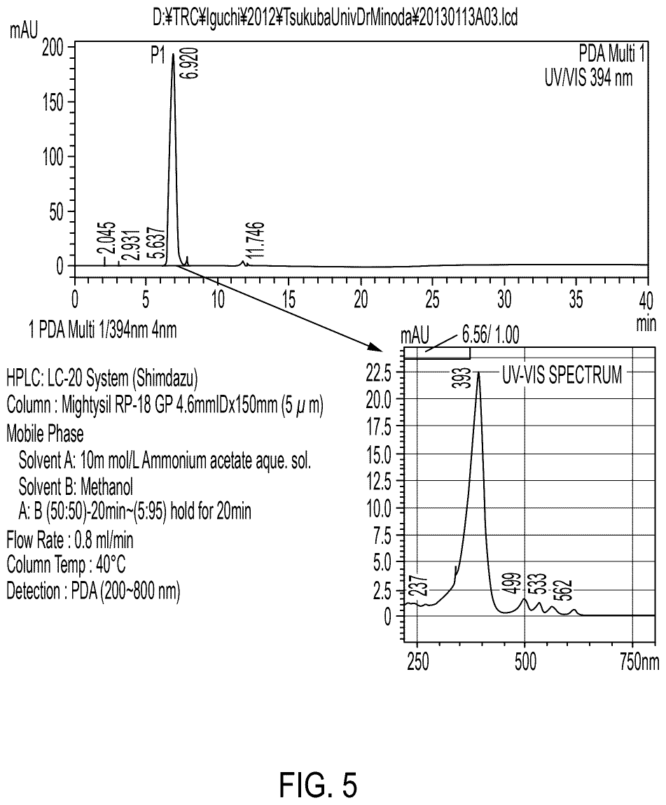

As a result, as shown in FIG. 2 and FIG. 3, a colorant having an absorption maximum at 400 nm and rare earth elements were included in large quantities. Furthermore, purification by HPLC was carried out based on the absorption maximum at 400 nm. Here, FIG. 4 is a diagram showing an ultraviolet-visible light absorption spectrum obtained before HPLC purification, and FIG. 5 is a diagram showing an ultraviolet-visible light absorption spectrum obtained after HPLC purification.

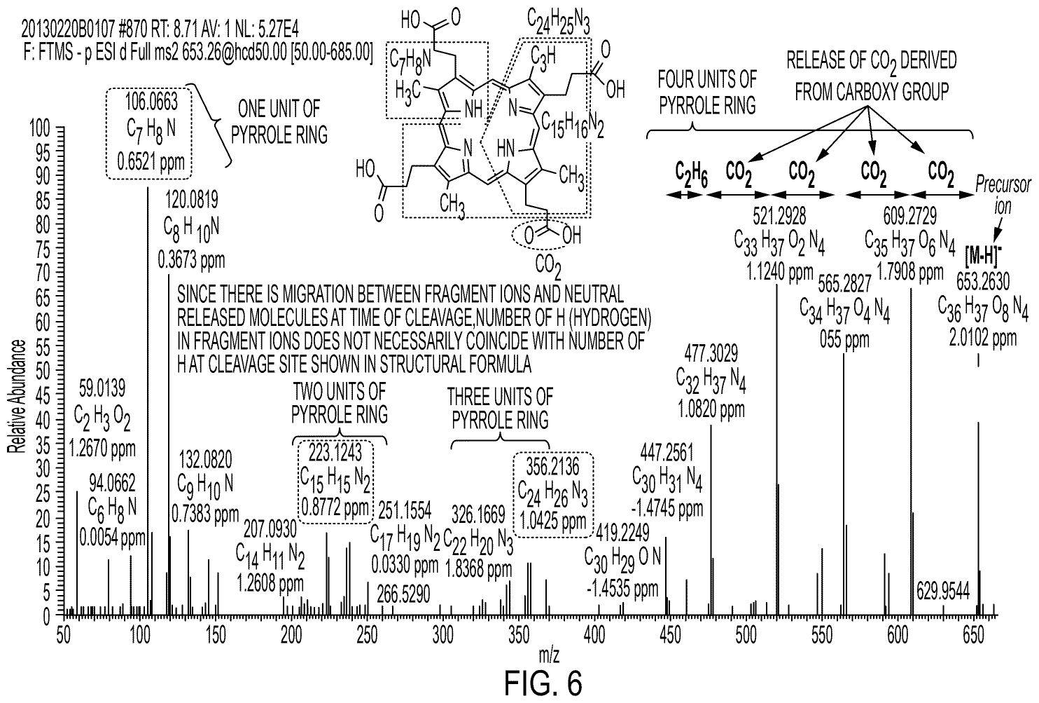

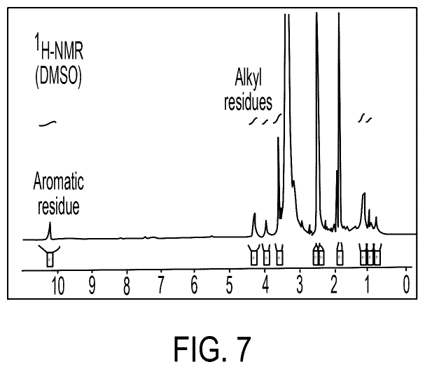

Then, an MS/MS analysis and a 1H-NMR analysis of the colorant purified by HPLC as shown in FIG. 5 were performed. Here, FIG. 6 is a diagram showing the MS/MS analysis results for the colorant purified by HPLC, and FIG. 7 is a diagram showing the 1H-NMR analysis results for the colorant purified by HPLC.

As shown in FIG. 6 and FIG. 7, as a result of performing the MS/MS analysis and 1H-NMR analysis, it was found that the colorant purified by HPLC was coproporphyrin (chemical structure thereof is shown below).

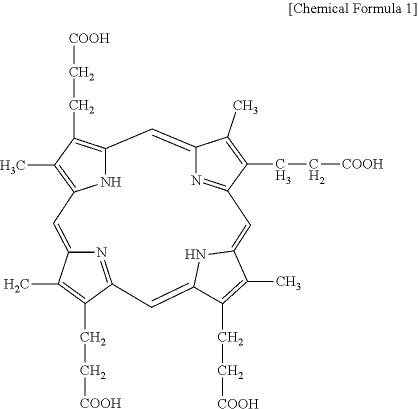 [SMILES: CC1=C(CCC(=O)O)c2cc3[nH]c(cc4nc(cc5[nH]c(cc1n2)c(CCC(=O)O)c5C)C(C)=C4CCC(=O)O)c(CCC(=O)O)c3C]

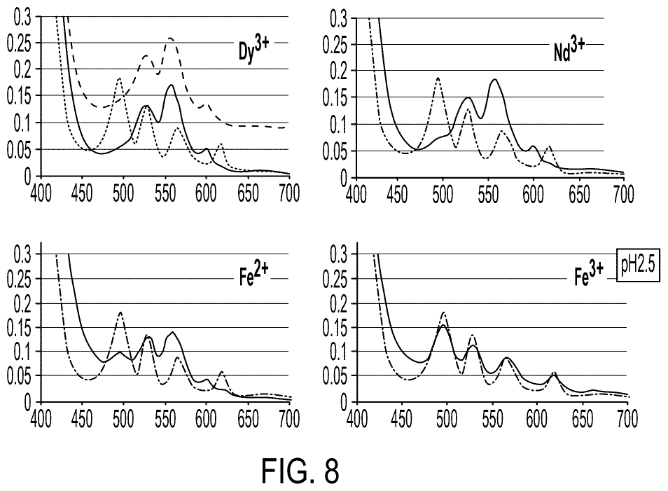

Generally, it is well known that a spectral shift in the visible light region occurs when a metal is chelated. Thus, Nd³⁺, Dy³⁺, Fe²⁺, and Fe³⁺ were added to a purified colorant, and any changes in the spectrum of the visible light region were observed. FIG. 8 is a diagram showing the spectra obtained at the time of no metal addition (HPLC purification product (coproporphyrin) of an ethyl acetate-extracted fraction: dotted lines in the diagram), at the time of metal addition of Nd³⁺, Dy³⁺, Fe²⁺, and Fe³⁺ (HPLC purification product (coproporphyrin of an ethyl acetate-extracted fraction+metals: solid lines in the diagram), and at the time of further addition of EDTA (HPLC purification product (coproporphyrin) of an ethyl acetate-extracted fraction+metals+EDTA: broken lines in the diagram).

As a result, as shown in FIG. 8, a shift in the spectrum of the visible light region was seen only with Nd³⁺, Dy³⁺, and Fe²⁺, and a shift was not observed with Fe³⁺. Furthermore, when EDTA as a metal chelator was added, the spectral shifts observed with Nd³⁺ and Fe²⁺ returned to the state before the addition of the metals, while the spectral shift observed with Dy³⁺ did not return to the original state.

Based on these results, it was confirmed that Nd³⁺ and Fe²⁺ were chelated by coproporphyrin. It was found that the bonding state of Dy³⁺ and coproporphyrin was not inhibited by the addition of EDTA, unlike the chelated state of Nd³⁺ and Fe³⁺, and a structural change in the porphyrin ring occurred as a result of the addition of EDTA. These results are considered to be caused by the difference in the stability constant of an EDTA complex between the various rare earth elements.

The experiment of bioleaching of Example 1 was carried out under acidic (pH 2.5) conditions. Since iron exists not in a divalent form (Fe²⁺) but in a trivalent form (Fe³⁺) under acidic conditions, it is speculated that coproporphyrin in the medium supernatant chelates more rare earth elements (Nd³⁺ and Dy³⁺) than iron (Fe³⁺).

From the results described above, the inventors of the present invention found that rare earth elements and iron can be separated by utilizing coproporphyrin under acidic conditions in which iron exists in a trivalent form. That is, the inventors found a method of selectively recovering rare earth elements such as lanthanoids or noble metals from a base metal mixture solution of iron and the like, under acidic conditions by using a porphyrin.

### Example 3

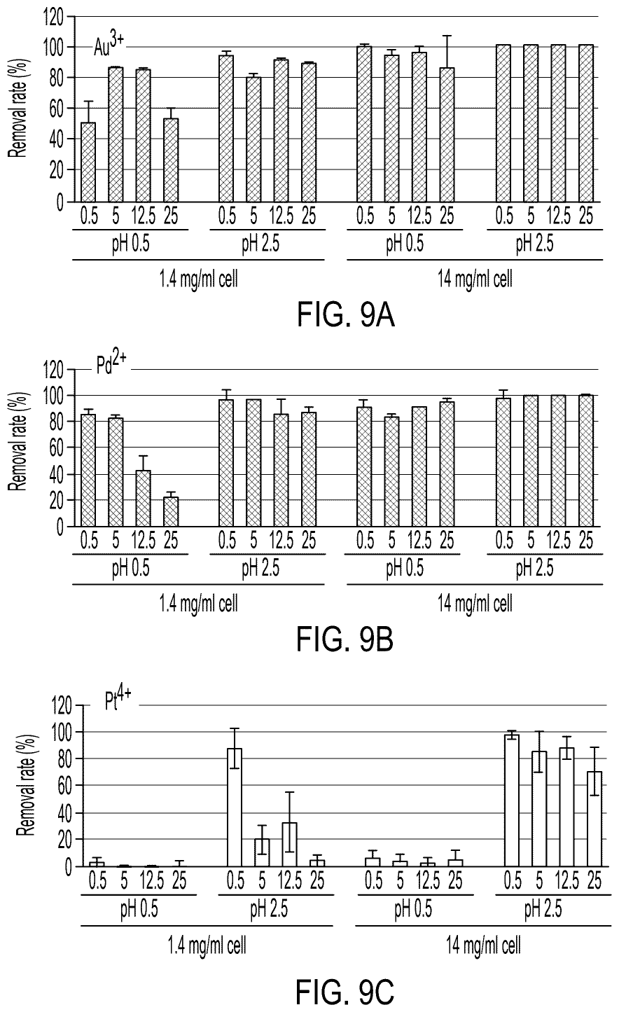

Example 3 relating to the recovery of noble metals by an alga belonging to the order Cyanidiales will be described below. In Example 3, the cell concentration and the acid concentration of a hydrochloric acid solution were changed, and then the recovery efficiencies for 0 to 25 ppm of gold, platinum, and palladium achieved by G. sulphuraria were investigated. FIG. 9A-FIG. 9C are diagrams of graphs showing the recovery efficiencies for gold, platinum, and palladium achieved by G. sulphuraria.

Regarding the acid concentration, the experiment was carried out using two kinds of solutions, namely, a 0.4 M hydrochloric acid solution (pH 0.5) and a 40 mM hydrochloric acid solution (pH 2.5). To these hydrochloric acid solutions, Au³⁺, Pd²⁺, and Pt⁴⁺ were added, and G. sulphuraria cells were cultured therein for 30 minutes. Regarding the cell density, the experiment was carried out with two kinds of densities, namely, 1.4 mg/ml and 14 mg/ml as dry weights.

After culturing, supernatant fractions and cells were separated by centrifugation. The metal concentrations in the supernatant fraction were determined by ICP-MS, and the percentage of each fraction was determined by subtracting the concentration obtainable as a control in the case of culturing without addition of cells from the concentration in each fraction. In addition, the concentrations of the hydrochloric acid solutions including Au³⁺, Pd²⁺, and Pt⁴⁺ without cell addition were as follows.

$$0.5 \pm 0.2 , 4.5 \pm 0.9 , 14 \pm 1.4 , 28 \pm 2.8 ( {Au}^{3 +} , pH 0.5 )$$

$$0.9 \pm 0. 3 , 2.9 \pm 0.1 , 7.6 \pm 0.8 , 16 \pm 3.6 ( {Au}^{3 +} , pH 2.5 )$$

$$0.4 \pm 0. 1 , 4.1 \pm 1.2 , 8.4 \pm 1.6 , 20 \pm 3.3 ( {Pd}^{2 +} , pH 0.5 )$$

$$0.3 \pm 0. 1 , 4.0 \pm 0.7 , 9.4 \pm 2.0 , 17 \pm 1. ( {Pd}^{2 +} , pH 2.5 )$$

$$0.6 \pm 0. 1 , 6.0 \pm 1.5 , 15 \pm 3.2 , 31 \pm 5.1 ( {Pt}^{4 +} , pH 0.5 )$$

$$0.6 \pm 0. 4 , 3.4 \pm 1.5 , 8.6 \pm 2.0 , 19 \pm 4.1 ( {Pt}^{4 +} , pH 2.5 )$$

(each value is the average value of three independent experiment values for each solution±SD value)

As a result, as shown in FIG. 9A-FIG. 9C, it was found that gold and palladium are recovered into cells with the highest efficiencies at pH 0.5 (0.4 M HCl). Furthermore, it was found that platinum is recovered into cells with high efficiency by increasing the amount of cells at a low acid concentration. Therefore, it was confirmed that noble metals at very low concentrations can be recovered with high efficiency by utilizing red algae such as the algae belonging to the order Cyanidiales.

### Example 4

Example 4 relating to the recovery efficiency in living cells (Living Cells) and dead cells (Freeze-thawed Cells) will be described below.

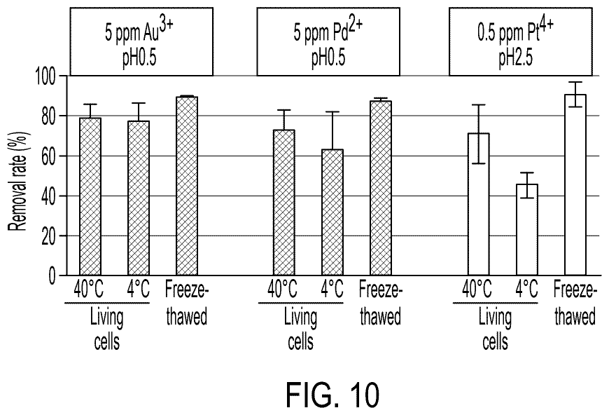

Here, FIG. 10 is a diagram of graphs showing the recovery efficiency in living cells (Living Cells) and dead cells (Freeze-thawed Cells) of G. sulphuraria. As shown in the diagram, living cells or dead cells were cultured in a 0.4 M hydrochloric acid solution (pH 0.5) containing 5 ppm of Au³⁺, a 0.4 M hydrochloric acid solution (pH 0.5) containing 5 ppm of Pd²⁺, or a 40 mM hydrochloric acid solution (pH 2.5) containing 0.5 ppm of Pt⁴⁺.

After culturing the cells at 40° C. or 4° C. for 30 minutes, a supernatant fraction was separated from the cells by centrifugation, and the concentrations were determined by ICP-MS. The percentage of each fraction was determined by subtracting the concentration obtainable as a control in the case of culturing without addition of cells from the concentration in each fraction. In addition, the concentrations of Au³⁺, Pd²⁺, and Pt⁴⁺ without cell addition were 2.5±0.6, 4.6±0.7, and 0.4±0.2, respectively (each value was the average value of three independent experiment results for each metal±SD value).

As a result, as shown in FIG. 10, the recovery efficiency was higher with dead cells than with living cells. In addition, for palladium and platinum, when the culture temperature for living cells was decreased to 4° C., the recovery efficiency decreased. There was no change in the case of gold. In this regard, it is generally known that biosorption is not affected by temperature; however, this is speculated to be because palladium and platinum are known to undergo endothermic reactions (Wang, J., Wei, J., and Li, J., 2015. Rice straw modified by click reaction for selective extraction of noble metal ions. Bioresour. Technol. 177, 182-187).

### Example 5

Subsequently, Example 5 relating to selective recovery in the presence of a plurality of metal ions will be described below.

A metal effluent dilution containing 70 ppm of iron, 360 ppm of copper, 5 ppm of platinum, 60 ppm of gold, 60 ppm of nickel, 6 ppm of tin, 18 ppm of palladium, and 12 ppm of zinc in aqua regia having an acid concentration of about 0.5 M was incubated for 30 minutes, with added cells of G. sulphuraria in an amount equivalent to 7 mg (+Cell), or without added cells (−Cell).

After culturing, the cells were caused to sediment by centrifugation, the concentrations of the respective metals in the supernatant were measured, and the removal rates were determined (following Table 2). The following table is a table showing the recovery efficiencies for Au³⁺ and Pd²⁺ from a metal effluent including a diluted aqua regia. Here, the aqua regia was produced from 57 ppm of Fe²⁺/³⁺+, 480 ppm Cu²⁺, 4 ppm of Pt⁴⁺, 53 ppm of Au³⁺, 46 ppm of Ni²⁺, 5 ppm of Sn²⁺, 12 ppm of Pd²⁺, 11 ppm of Zn²⁺, and 0.56 M acid.

TABLE 2

| | Fe^{2+/3+} | Cu²⁺ | Pt⁴⁺ | Au³⁺ | Ni²⁺ | Sn²⁺ | Pd²⁺ | Zn²⁺ |
|---|---|---|---|---|---|---|---|---|
| | (ppm) | (ppm) | (ppm) | (ppm) | (ppm) | (ppm) | (ppm) | (ppm) |
| −Cell | 88 ± 8.6 | 379 ± 46 | 5.8 ± 0.6 | 61 ± 9 | 59 ± 7.5 | 6.5 ± 0.8 | 18 ± 2.0 | 12 ± 1.4 |
| +Cell | 63 ± 11 | 358 ± 60 | 4.7 ± 0.7 | 5.9 ± 2.8 | 58 ± 9.4 | 5.5 ± 0.8 | 0.9 ± 0.2 | 12 ± 1.9 |
| Removal | 8% | 6% | 19% | 90% | 0.30% | 16% | 95% | 6% |
| ratio | | | | | | | | |

As a result, only gold and palladium were recovered into the cells with high efficiency. Furthermore, gold and palladium could not be recovered in the aqua regia solution having a high acid concentration (following Table 3). The following table is a table showing the recovery efficiencies for Au³⁺ and Pd²⁺ from a metal effluent including an aqua regia having high acidity. Here, the aqua regia was produced from 570 ppm of Fe²⁺/³⁺, 4800 ppm of Cu²⁺, 40 ppm of Pt⁴⁺, 530 ppm of Au³⁺, 460 ppm of Ni²⁺, 50 ppm of Sn²⁺, 120 ppm of Pd²⁺, 110 ppm of Zn²⁺, and 5.6 M acid.

TABLE 3

| | Elements | | | | | | | |
|---|---|---|---|---|---|---|---|---|
| | Fe^{2+/3+} | Cu²⁺ | Pt⁴⁺ | Au³⁺ | Ni²⁺ | Sn²⁺ | Pd²⁺ | Zn²⁺ |
| Sample | ppm | ppm | ppm | ppm | ppm | ppm | ppm | ppm |
| −Cell | 529 ± 24 | 3364 ± 304 | 42 ± 15 | 464 ± 25 | 476 ± 39 | 40.2 ± 7.8 | 135 ± 10 | 182 ± 40 |
| +Cell | 475 ± 28 | 3237 ± 235 | 26 ± 2.3 | 406 ± 69 | 428 ± 9.6 | 27.8 ± 2.0 | 116 ± 6.3 | 161 ± 13 |
| (Removal | (10%) | (3.8%) | (3.8%) | (13%) | (10%) | (31%) | (14%) | (12%) |
| rate %) | | | | | | | | |

Furthermore, even when the gold concentration was about 580 ppm, a recovery efficiency of 60% was maintained (following Table 4). The following table is a table showing the recovery efficiencies achieved by G. sulphuraria cells from a metal effluent including an aqua regia containing Au³⁺ at a high concentration. Here, the aqua regia was produced from 70 ppm of Fe²⁺/³⁺, 120 ppm of Cu²⁺, 3 ppm of Pt⁴⁺, 577 ppm of Au³⁺, 210 ppm of Ni²⁺, 14 ppm of Sn²⁺, and 0.43 M acid.

TABLE 4

| | Fe^{2+/3+} | Cu²⁺ | Pt⁴⁺ | Au³⁺ | Ni²⁺ | Sn²⁺ |
|---|---|---|---|---|---|---|
| | (ppm) | (ppm) | (ppm) | (ppm) | (ppm) | (ppm) |
| −Cell | 86 ± 14 | 107 ± 44 | 4.5 ± 0.6 | 580 ± 57 | 262 ± 48 | 24 ± 3.5 |
| +Cell | 63 ± 7.6 | 73 ± 11 | 3.7 ± 0.3 | 199 ± 72 | 223 ± 19 | 18 ± 1.3 |
| Removal rate | 26% | 28% | 17% | 66% | 14% | 23% |

As a result of the above, it was confirmed that even in a case in which a plurality of metal ions exist in large quantities, only noble metal ions can be selectively recovered.

### Example 6

Example 6 relating to the elution of the noble metal ions recovered into an algal body will be described below.

Similarly to Example 5 described above, a metal effluent dilution containing 70 ppm of iron, 360 ppm of copper, 5 ppm of platinum, 60 ppm of gold, 60 ppm of nickel, 6 ppm of tin, 18 ppm of palladium, and 12 ppm of zinc in an aqua regia having an acid concentration of about 0.5 M was incubated for 15 minutes with added cells of G. sulphuraria in an amount equivalent to 7 mg.

Then, the cells that had recovered 57 ppm of gold and 15 ppm of palladium were incubated for 30 minutes in an elution solution indicated in the following Table 5. The following Table 5 is a table showing the elution of Au³⁺ and Pd²⁺ from the G. sulphuraria cells that had recovered 59±7 ppm of Au³⁺ and 15±1 ppm of Pd²⁺ from a diluted metal effluent. Regarding the cells, cells that had been incubated for 15 minutes in a diluted metal effluent containing 57 ppm of Fe²⁺/³⁺, 480 ppm of Cu²+, 4 ppm of Pt⁴⁺, 53 ppm of Au³⁺, 46 ppm of Ni²⁺, 5 ppm of Sn²⁺, 12 ppm of Pd²⁺, 11 ppm of Zn²⁺, and 0.56 M acid were used (each value represents the average value±S. E. value).

TABLE 5

| | Au³⁺ | Pd²⁺ | Fe^{2+/3+} | Cu²⁺ | Pt⁴⁺ | Ni²⁺ | Sn²⁺ | Zn²⁺ |
|---|---|---|---|---|---|---|---|---|
| Elution solution | ppm | ppm | ppm | ppm | ppm | ppm | ppm | ppm |
| 0.4M HCl | 2.4 ± 0.8 | 0.0 ± 0.0 | 2.2 ± 2.2 | 11.6 ± 0.1 | ND | ND | 0.2 ± 0.0 | ND |
| 0.2M NH₄Br, | 29.2 ± 2.3 | 11.6 ± 0.7 | ND | 3.8 ± 3.8 | ND | ND | ND | ND |
| 2.8% NH₃ (pH 11) | | | | | | | | |
| 0.2M NH₄Cl, | 28.2 ± 0.3 | 11.3 ± 0.3 | ND | ND | ND | ND | ND | ND |
| 2.8% NH₃ (pH 11) | | | | | | | | |
| 0.1M KOH | 3.9 ± 1.6 | 0.4 ± 0.1 | ND | 3.8 ± 3.8 | ND | ND | 0.4 ± 0.1 | ND |
| 1M Thiourea, | 46.2 ± 6.4 | 13.4 ± 1.2 | 5.5 ± 2.8 | 11.6 ± 0.1 | 0.6 ± 0.0 | ND | ND | ND |
| 0.1M HCl | | | | | | | | |

As a result, 48% of gold ions and 70% of palladium ions were eluted into the solution. Incorporation of iron or copper was significantly suppressed compared to the existing methods of utilizing thiourea (above Table 5). Furthermore, it was found that the recovery efficiency significantly decreases when ammonia only is used, or ammonium ions only are used (following Table 6). The following table is a table showing the various metal concentrations and recovery rates in an elution solution in which the cells that had recovered Au³⁺ and Pd²⁺ were incubated for 30 minutes.

TABLE 6

| Concentration of | | Concentration and recovery rate in the | | | |
|---|---|---|---|---|---|
| Au³⁺ and Pd²⁺ | | elution solution after the elution | | | |
| retained by the cells | Elution | Au³⁺ | Pd²⁺ | Au³⁺ | Pd²⁺ |
| before the elution | solution | ppm | | % | |
| 121 ppm Au³⁺, | HCl | 3.3 ± 2.9 | 0 ± 0 | 2.7 | 0 |
| 36 ppm Pd²⁺ | (pH 0.5) | | | | |
| 121 ppm Au³⁺, | 2.8% | 7.6 ± 0.3 | 2.7 ± 0.3 | 6.2 | 7.7 |
| 36 ppm Pd²⁺ | NH₃ | | | | |
| 121 ppm Au³⁺, | 14% | 11 ± 1.3 | 3.6 ± 0.6 | 9 | 10.2 |
| 36 ppm Pd²⁺ | NH₃ | | | | |
| 65 ppm Au³⁺, | 0.2M | 5.6 ± 0.2 | 0 ± 0 | 8.7 | 0 |
| 18 ppm Pd²⁺ | NH₄Cl | | | | |
| | (pH 3) | | | | |
| 65 ppm Au³⁺, | 0.2M | 12 ± 2.1 | 2.4 ± 0.2 | 18 | 13 |
| 18 ppm Pd²⁺ | NH₄Cl | | | | |
| | (pH 7) | | | | |
| 65 ppm Au³⁺, | 0.2M | 26 ± 0.8 | 8.9 ± 0.5 | 39 | 49 |
| 18 ppm Pd²⁺ | NH₄Cl | | | | |
| | (pH 11) | | | | |

As disclosed in this Example 6, it was confirmed that the recovery into the algal body was achieved within 15 minutes, the extraction from the algal body was achieved within 30 minutes, and treatment can be achieved in a short time period. Furthermore, it was confirmed by this Example 6 that noble metals can be selectively recovered from an aqua regia solution using an alga belonging to the order Cyanidiales by adjusting the acid concentration of the solution to be about 0.5 M. Furthermore, it was found that a noble metal can be extracted and purified as a complex by utilizing a mixed liquid of aqueous ammonia and an ammonium salt (ammonium chloride, ammonium sulfate, ammonium carbonate, ammonium bromide, or the like).

From this, it was confirmed that compared to a method of utilizing thiourea under acidic conditions, incorporation of other metals can be suppressed, and purity can be increased. It was confirmed that since complexes that are also used for solvent extraction of noble metals are used for elution and purification of noble metals, application of the method to conventional chemical processes or production processes is facilitated, and the method is superior to conventional methods from the viewpoint of economic efficiency or from an environmental viewpoint.

Here, the experimental results obtained in the case of using chlorella instead of red alga G. sulphuraria as a control experiment are shown in the following Table 7. The following table is a table showing the recovery efficiencies for Au³⁺ and Pd²⁺ from a metal effluent including a diluted aqua regia using chlorella cells. Meanwhile, the aqua regia was produced from 57 ppm of Fe²⁺/³⁺, 480 μm of Cu²⁺, 4 ppm of Pt⁴⁺, 53 ppm of Au³⁺, 46 ppm of Ni²⁺, 5 ppm of Sn²⁺, 12 ppm of Pd²⁺, 11 ppm of Zn²⁺, and 0.56 M acid (each value is the average value±S. D. value).

TABLE 7

| | Fe^{2+/3+} | Cu²⁺ | Pt⁴⁺ | Au³⁺ | Ni²⁺ | Sn²⁺ | Pd²⁺ | Zn²⁺ |
|---|---|---|---|---|---|---|---|---|
| | (ppm) | (ppm) | (ppm) | (ppm) | (ppm) | (ppm) | (ppm) | (ppm) |
| −Cell | 52 ± 13 | 314 ± 130 | 48 ± 0.6 | 69 ± 9 | 44 ± 84 | 5.7 ± 0.8 | 16.2 ± 2.4 | 15 ± 7 |
| +Cell | 64 ± 17 | 339 ± 60 | 49 ± 1.0 | 41 ± 17 | 47 ± 15 | 5.6 ± 1.1 | 3.4 ± 1.4 | 19 ± 12 |
| Removal rate | — | — | — | 40% | — | 2% | 79% | — |

Since aqua regia has very high metal solubility, even in the case of chlorella that has been reported to recover gold ions with high efficiency in a hydrochloric acid solution, the recovery efficiency for gold ions and palladium ions from an aqua regia solution are 40% and 79%, respectively as shown in the above table, while in the present method, the recovery efficiencies are 90% or higher (Table 7 described above). Here, the following Table 8 is a table showing the recovery rates from a metal effluent including an aqua regia containing Au³⁺ at a high concentration. In addition, the cells were incubated for 15 minutes in a diluted metal effluent containing 57 ppm of Fe²⁺/³⁺, 480 ppm of Cu²⁺, 4 ppm of Pt⁴⁺, 53 ppm of Au³⁺, 46 ppm of Ni²⁺, 5 ppm of Sn²⁺, 12 ppm of Pd²⁺, 11 ppm of Zn²⁺, and 0.56 M acid (each value is the average value±S. E. value).

TABLE 8

| | Au³⁺ | Pd²⁺ | Fe^{2+/3+} | Cu²⁺ | Pt⁴⁺ | Ni²⁺ | Sn²⁺ | Zn²⁺ |
|---|---|---|---|---|---|---|---|---|
| Elution solution | ppm | ppm | ppm | ppm | ppm | ppm | ppm | ppm |
| 0.4M HCl | 4.6 ± 1.4 | 0.2 ± 0.1 | ND | 3.8 ± 3.8 | ND | ND | 0.1 ± 0.1 | ND |
| 0.2M NH₄Cl, 2.8% NH₃ (pH 11) | 8.7 ± 1.3 | 8.2 ± 0.1 | ND | 7.5 ± 3.8 | ND | ND | ND | ND |
| 1M Thiourea, 0.1M HCl | 11.9 ± 4.2 | 10.0 ± 0.7 | 2.6 ± 2.6 | 3.8 ± 3.8 | 0.1 ± 0.0 | ND | ND | ND |

### Example 7

Subsequently, Example 7 relating to recovery and nanoparticulation by reduction of gold ions at a low concentration by G. sulphuraria will be described below.

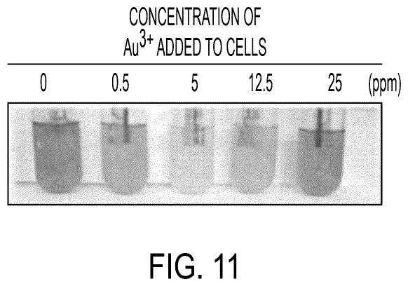

First, gold ions at a concentration of 0 to 25 ppm were added to cells of G. sulphuraria. FIG. 11 is a diagram (photograph as a substitute for a diagram) showing the concentration of gold ions added to the cells and the color changes in the culture fluid. As a result, G. sulphuraria recovered 25 ppm or less of gold ions with an efficiency of 90% or higher. The following Table 9 is a table showing the concentration of gold ions added to the cells and the recovery rate (%) into the cells.

TABLE 9

| | Au³⁺ concentration in supernatant | | |
|---|---|---|---|
| Concentration of Au³⁺ | after 30 minutes of addition | | Recovery |
| added to cells | (ppm) | | efficiency |
| (ppm) | −Cell | +Cell | (%) |
| 0 | 0.03 ± 0.004 | 0.03 ± 0.008 | — |
| 0.5 | 0.37 ± 0.008 | 0.03 ± 0.004 | 91.9 |
| 5 | 4.01 ± 0.134 | 0.03 ± 0.003 | 99.3 |
| 12.5 | 13.1 ± 0.08 | 0.07 ± 0.014 | 99.7 |
| 25 | 26.3 ± 0.08 | 0.07 ± 0.014 | 99.8 |

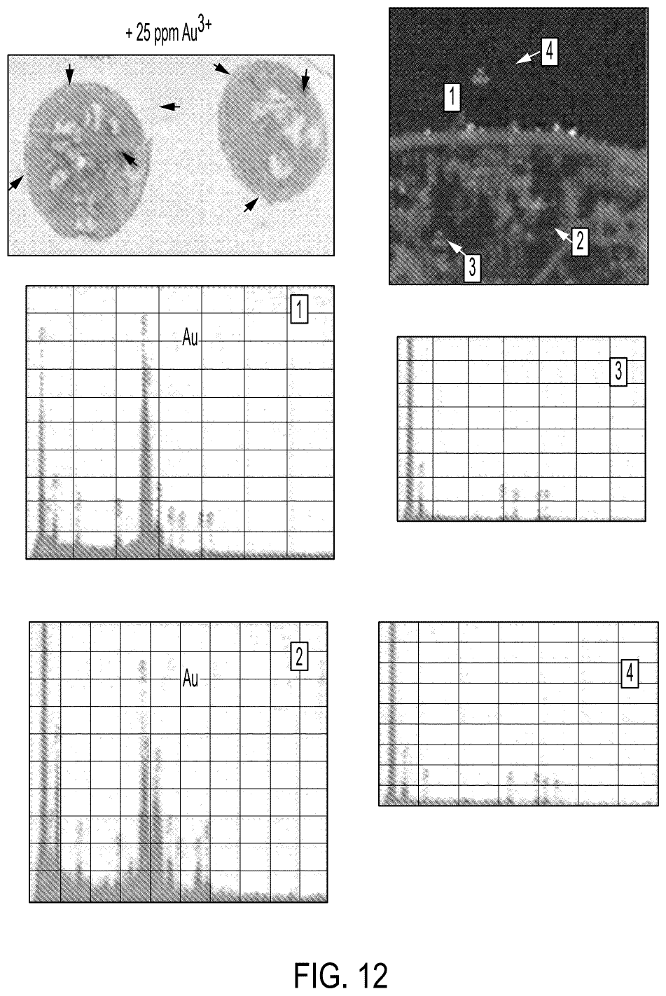

Furthermore, it was found that at 25 ppm, the cells of G. sulphuraria not only recover gold ions but also reduce the gold ions thus recovered, and form reddish purple gold nanoparticles mainly in the cell surface layer. Here, FIG. 12 is a diagram (photographs as a substitute for diagrams) showing the positions of gold nanoparticles in a microscopic image of cells and the Au composition analysis results obtained by TEM-EDS.

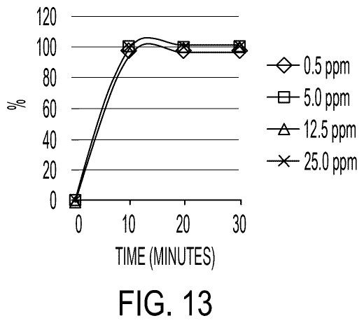

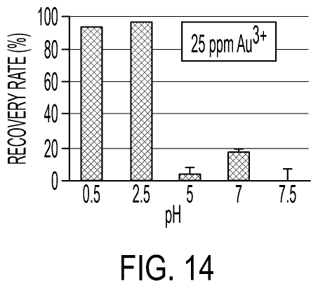

Furthermore, FIG. 13 is a diagram of a graph showing the recovery rates at a gold ion concentration of 0.5 to 25.0 ppm and the incubation time. As shown in FIG. 13, the recovery of gold ions reached 100% within 10 minutes. Furthermore, this recovery of gold ions occurred under acidic conditions. Here, FIG. 14 is a diagram of a graph showing pH-dependent recovery rates of gold ions into cells.

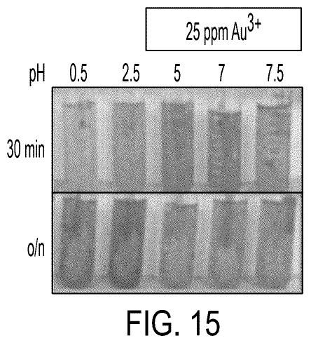

Furthermore, here, FIG. 15 is a diagram (photograph as a substitute for a diagram) showing the color changes in the culture fluid depending on pH and the incubation time. As shown in FIG. 15, at the time point of 30 minutes where the recovery of gold ions reached 100%, the color of the culture fluid was yellow, and the color changed to reddish purple after incubation overnight (o/n: over night).

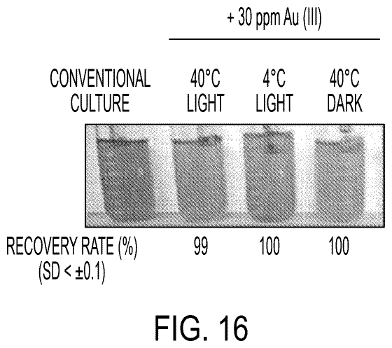

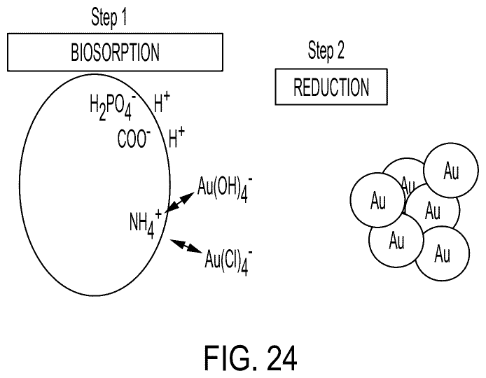

According to these results, it was found that reduction and recovery of gold ions by G. sulphuraria involve two steps of a rapid recovery step that takes 10 minutes or less (recovery process) and a reduction step that requires several hours (reduction process) (see FIG. 24). In the former recovery step, the recovery of gold ions into a cell surface layer is based on biosorption (see Examples 3 to 6 described above). Here, FIG. 16 is a diagram (photograph as a substitute for a diagram) showing the color changes in the culture fluid occurring in a case in which incubation was performed at various temperatures in a dark place and a bright place.

As shown in FIG. 16, it was found that in the recovery step, light and temperature do not have any influence; however, in the reduction step, reduction of gold ions requires light and temperature.

### Example 8

Example 8 relating to photoreduction of gold ions by porphyrins (pheophytin and coproporphyrin) will be described below.

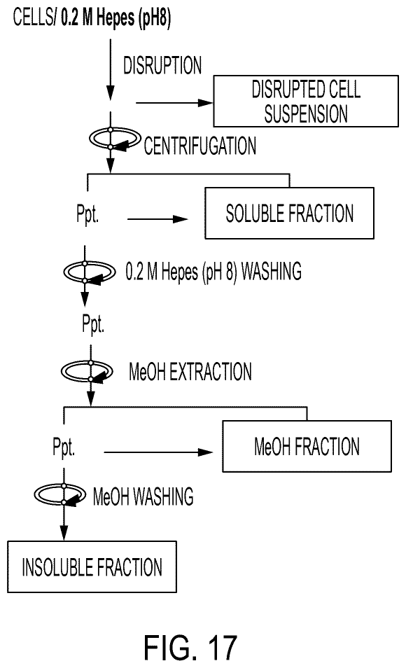

Subsequently, based on the clue that reduction of gold ions depends on light and temperature, an investigation was conducted on the substance related to the reduction of gold ions in the latter reduction step. Here, FIG. 17 is a flow chart showing the method for preparing various cell fractions.

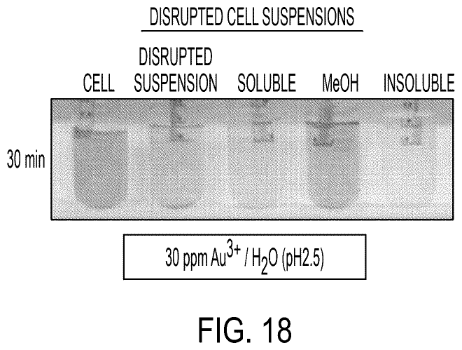

As shown in FIG. 17, cells were fractionated, gold ions were added to various fractions, and incubation was performed for 30 minutes. Here, FIG. 18 is a diagram (photograph as a substitute for a diagram) showing color changes occurring after adding gold ions to various cell fractions and culturing the cells for 30 minutes. As shown in FIG. 18, after the incubation, reduction of gold ions occurred in a methanol (MeOH)-extracted fraction, and gold nanoparticles were observed.

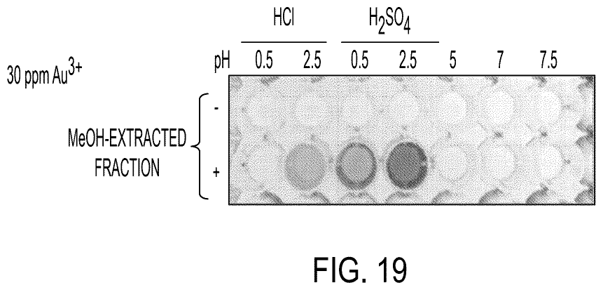

Next, the pH was changed, and incubation of the MeOH-extracted fraction and gold ions was performed. Here, FIG. 19 is a diagram (photograph as a substitute for a diagram) showing the results obtained by incubating the methanol-extracted fraction by changing the pH.

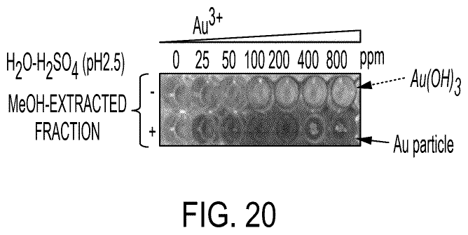

As shown in FIG. 19, reduction of gold ions occurred only under acidic conditions. Thus, the gold ion concentration was increased, and incubation was performed. FIG. 20 is a diagram (photograph as a substitute for a diagram) showing the results of incubating the methanol-extracted fraction while changing the gold ion concentration. As shown in FIG. 20, gold-colored structures having a size larger than reddish purple gold nanoparticles were observed along with an increase in the concentration of gold ions. In addition, in the control (upper row in FIG. 20: MeOH-extracted fraction-) in which the MeOH-extracted fraction was not added and methanol and gold ions were simply mixed, a yellowish brown precipitate, which was considered to be gold hydroxide, was observed.

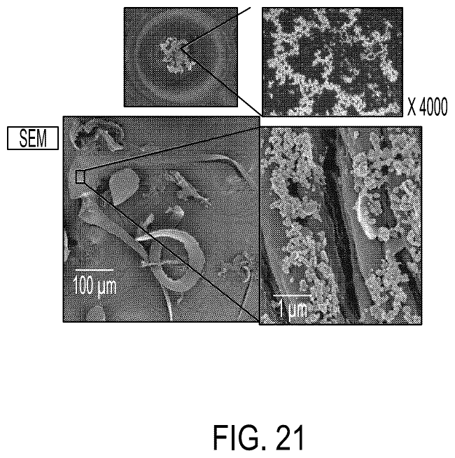

Subsequently, the gold-colored structures produced by incubation of the MeOH-extracted fraction and gold ions at a high concentration were observed with a CCD camera and a SEM. FIG. 21 is a diagram (photographs as a substitute for diagrams) showing CCD camera images and SEM images of the gold-colored structures produced by incubation of the MeOH-extracted fraction and gold ions at a high concentration.

As shown in FIG. 21, ribbon-shaped structures were observed, and it was found that the surface thereof was covered with fine particles that were considered to be gold particles. As described above in connection with FIG. 2 and FIG. 5, G. sulphuraria releases coproporphyrin out of the cell. Furthermore, chlorophyll that exists in a large quantity in the cell and is extracted with MeOH is such that under acidic conditions, magnesium as the central metal comes off, and the resultant exists as pheophytin. Furthermore, photoreduction of Fe³⁺ to Fe²⁺ by porphyrin in methanol is known (Bartocci et al., 1980, J. Am. Chem. Soc., 102, 7067-7072).

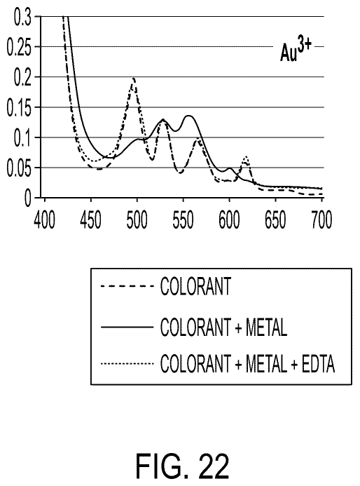

Here, FIG. 22 is a diagram of a graph showing spectral shifts at the visible light portion obtained in a case in which gold ions were added to coproporphyrin. As shown in FIG. 22, when gold ions are added to coproporphyrin that has been purified from the outside of the cells of G. sulphuraria, spectral shifts occur at the visible light portion, and coproporphyrin chelates gold ions.

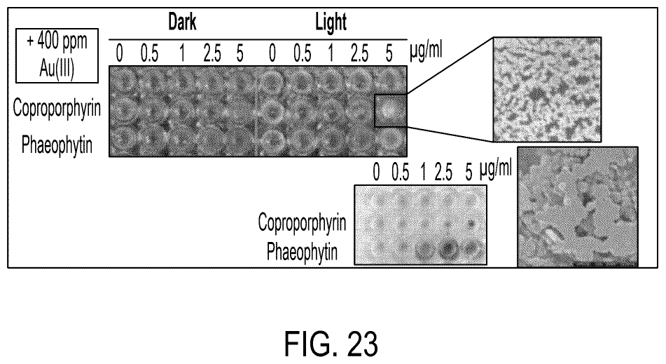

Here, FIG. 23 is a diagram (photographs as a substitute for diagrams) showing the results of adding gold ions to preparations of coproporphyrin and pheophytin and incubating the preparations overnight. As shown in FIG. 23, it was found that when light is radiated in the presence of coproporphyrin or pheophytin, reduction of gold ions and formation of gold particles are promoted.

Photoreduction of gold ions by porphyrins occurred with about 50 μE of light, and strong light such as laser light was not needed. From these results, it was found that nanoparticulation of gold ions by photoreduction by porphyrins such as coproporphyrin and pheophytin occurs, and gold nanoparticles having different sizes are formed as a result of the difference in the type of porphyrin.

Based on these findings, when porphyrins are used, recovery and purification of gold ions with high purity can now be carried out by nanoparticulation by reduction of gold ions at concentrations lower than conventional cases. Furthermore, it was found that since porphyrins have selectivity, even under conditions in which a plurality of metal ions exist in large quantities, only gold ions can be selectively purified as gold particles with high purity.

### Example 9

Here, Example 9 of comparing a material derived from an alga belonging to the order Cyanidiales and chlorella will be described below.

As a result of an experiment, a cell surface layer of an alga belonging to the order Cyanidiales exhibited superior adsorption and desorption of gold (metal), compared to a surface layer of algae of the prior art technologies concerning bioleaching and biosorption using chlorella (for example, Japanese Examined Patent Publication No. S62-500931) (seethe following Table 10). Furthermore, it was found, based on the Examples described above, that unlike the priority art technologies related to red algae (Japanese Unexamined Patent Publication No. 2013-67826), the function is achieved even with dead cells or a cell surface layer only. Thereby, it was confirmed that a cell surface layer or an artificial material simulating a cell surface layer can be processed into or supplied to a form that can be more easily utilized as a material derived from an alga belonging to the order Cyanidiales.

TABLE 10
Table Comparison of recovery efficiencies for gold ions from
metal effluent, obtained by utilizing Galdieria and Chlorella

| | Galdieria | Chlorella |
|---|---|---|
| | (ppm) | (ppm) |
| −Cell | 61 ± 9 | 69 ± 9 |
| +Cell | 5.9 ± 2.8 | 41 ± 17 |
| Removal rate | 90% | 40% |

Hereinafter, the present invention will be described in more detail with reference to examples and comparative examples, but the present invention is not limited thereto.

In the following description, “recovery rate” refers to a value calculated by Formula (2). In addition, the “cell non-addition test” in Formula (2) refers to a test conducted without adding any of the dry powder produced from live cells of G. sulphuraria and cells of G. sulphuraria. In addition, the “cell addition test” in Formula (2) refers to a test conducted by adding the dry powder produced from the live cells of G. sulphuraria or the cells of G. sulphuraria.

$$\begin{matrix}[ Formula 2 ] & \\ Recovery rate ( \% ) = ( metal concentration of supernatant in cell non - addition test ) - ( metal concentration of supernatant in cell addition test ) / ( metal concentration of supernatant in cell addition test ) \times 100 & ( 2 )\end{matrix}$$

### Example 10

As Example 10, a selective recovery test of gold and palladium from a hydrochloric acid solution (0.5 to 6 M) was conducted using a spray-dried sample.

### (Production of Spray-Dried Sample)

Cells of G. sulphuraria grown to the stationary phase were recovered by centrifugation. Next, using a spray dryer, a spray-dried sample (dry powder) was produced from the recovered cells under the conditions of an inlet temperature of 120 degrees Celsius, an outlet temperature of 77 to 82 degrees Celsius, a hot air flow rate of 0.6 m³/min, and a spray pressure of 100 kPa. In the following description, the “spray-dried sample” refers to a sample produced according to the above-described method.

### (Test Nos. 1-1 and 1-2)

First, 10 mg of the spray-dried sample was added to 1 ml of a 0.5 M hydrochloric acid solution containing 5 ppm each of Au³⁺, Pd²⁺, and Cu²⁺, and stirred at room temperature for 15 minutes. Next, the hydrochloric acid solution was centrifuged (12,000 rpm, 1 minute), and the metal concentration of the obtained supernatant was measured using ICP-MS (Test No. 1-2).

In addition, as a control test, the same test as Test No. 1-2 was conducted except that the spray-dried sample was not added, and the metal concentration of the supernatant was measured (Test No. 1-1). Next, the recovery rate in Test No. 1-2 was calculated from the difference between the metal concentrations of Test No. 1-1 and Test No. 1-2 measured above. The measured metal concentration and the recovery rate are shown in Table 11. The metal concentrations and the recovery rates shown in Table 11 are the mean value of three independent tests±SD (standard deviation). In Tables 11 to 18 below, powder “+” indicates that the spray-dried sample or a freeze-dried sample was added, and powder “−” indicates that no spray-dried sample or freeze-dried sample was added.

### (Test Nos. 1-3 to 1-14)

Test Nos. 1-3 to 1-14 were conducted in the same manner as in Test No. 1-1 or 1-2 except that the concentration of the hydrochloric acid solution was changed to the concentration shown in Table 11. For each test, the measured metal concentration and the recovery rate are shown in Table 11.

TABLE 11

| | HCl | | Metal concentration of | | | | |
|---|---|---|---|---|---|---|---|
| Test | concentration | | supernatant (ppm) | | | Recovery rate (%) | |
| No. | (mol/L) | Powder | Au³⁺ | Pd²⁺ | Cu²⁺ | Au³⁺ | Pd²⁺ |
| 1-1 | 0.5 | − | 2.24 ± 0.54 | 2.24 ± 0.56 | 2.24 ± 0.58 | — | — |
| 1-2 | 0.5 | + | 0.25 ± 0.10 | 0.02 ± 0.03 | 2.84 ± 0.07 | 93 ± 3.7 | 100 ± 0.6 |
| 1-3 | 1.0 | − | 2.41 ± 0.54 | 2.03 ± 0.48 | 3.61 ± 0.44 | — | — |
| 1-4 | 1.0 | + | 0.09 ± 0.03 | 0.01 ± 0.01 | 3.04 ± 0.35 | 98 ± 1.1 | 100 ± 0.3 |
| 1-5 | 2.0 | − | 2.37 ± 0.53 | 2.02 ± 0.42 | 3.52 ± 0.57 | — | — |
| 1-6 | 2.0 | + | 0.08 ± 0.03 | 0.01 ± 0.01 | 3.07 ± 0.35 | 98 ± 0.8 | 100 ± 0.2 |
| 1-7 | 3.0 | − | 2.32 ± 0.67 | 2.04 ± 0.27 | 3.54 ± 0.16 | — | — |
| 1-8 | 3.0 | + | 0.07 ± 0.03 | 0.01 ± 0.01 | 3.21 ± 0.44 | 98 ± 0.7 | 100 ± 0.2 |
| 1-9 | 4.0 | − | 3.26 ± 2.27 | 3.40 ± 1.64 | 4.57 ± 2.22 | — | — |
| 1-10 | 4.0 | + | 0.08 ± 0.02 | 0.01 ± 0.01 | 4.01 ± 1.76 | 98 ± 0.9 | 100 ± 0.2 |
| 1-11 | 5.0 | − | 3.14 ± 2.36 | 3.36 ± 1.75 | 4.45 ± 2.26 | — | — |
| 1-12 | 5.0 | + | 0.08 ± 0.02 | 0.01 ± 0.01 | 3.97 ± 1.77 | 98 ± 0.8 | 100 ± 0.2 |
| 1-13 | 6.0 | − | 2.38 ± 0.59 | 2.01 ± 0.27 | 3.59 ± 0.42 | — | — |
| 1-14 | 6.0 | + | 0.09 ± 0.03 | 0.01 ± 0.01 | 3.13 ± 0.41 | 98 ± 0.8 | 100 ± 0.3 |

### Example 11

As Example 11, a selective recovery test of gold and palladium from a hydrochloric acid solution (0.5 to 6 M) was conducted using a freeze-dried sample.

### (Test Nos. 2-1 and 2-2)

Cells of G. sulphuraria grown to the stationary phase were recovered by centrifugation. Next, the recovered cells were frozen by liquid nitrogen and subjected to a vacuum freeze-drying treatment, thereby producing a freeze-dried sample (dry powder). Next, 10 mg of the freeze-dried sample produced above was added to 1 ml of a 0.5 M hydrochloric acid solution containing 5 ppm each of Au³⁺, Pd²⁺, and Cu²⁺, and stirred at room temperature for 15 minutes. Next, the hydrochloric acid solution was centrifuged (12,000 rpm, 1 minute), and the metal concentration of the obtained supernatant was measured using ICP-MS (Test No. 2-2).

In addition, as a control test, the same test as Test No. 2-2 was conducted except that the freeze-dried sample was not added, and the metal concentration of the supernatant was measured (Test No. 2-1). Next, the recovery rate in Test No. 2-2 was calculated from the difference between the metal concentrations of Test No. 2-1 and Test No. 2-2 measured above. The measured metal concentration and the recovery rate are shown in Table 12. The metal concentrations and the recovery rates shown in Table 12 are the mean value of three independent tests±SD (standard deviation).

### (Test Nos. 2-3 to 2-14)

Test Nos. 2-3 to 2-14 were conducted in the same manner as in Test No. 2-1 or 2-2 except that the concentration of the hydrochloric acid solution was changed to the concentration shown in Table 12. For each test, the measured metal concentration and the recovery rate are shown in Table 12.

TABLE 12

| | HCl | | Metal concentration of | | | | |
|---|---|---|---|---|---|---|---|
| Test | concentration | | supernatant (ppm) | | | Recovery rate (%) | |
| No. | (mol/L) | Powder | Au³⁺ | Pd²⁺ | Cu²⁺ | Au³⁺ | Pd²⁺ |
| 2-1 | 0.5 | − | 4.58 ± 0.45 | 4.15 ± 0.73 | 4.71 ± 0.76 | — | — |
| 2-2 | 0.5 | + | 0.01 ± 0.01 | 0.07 ± 0.05 | 4.61 ± 0.68 | 99.67 ± 0.58 | 98.10 ± 0.79 |
| 2-3 | 1 | − | 4.28 ± 1.13 | 3.11 ± 0.59 | 3.37 ± 0.64 | — | — |
| 2-4 | 1 | + | 0.08 ± 0.10 | 0.01 ± 0.00 | 3.36 ± 2.89 | 97.78 ± 2.12 | 99.23 ± 0.39 |
| 2-5 | 2 | − | 4.05 ± 0.12 | 3.28 ± 0.34 | 3.70 ± 0.40 | — | — |
| 2-6 | 2 | + | 0.16 ± 0.14 | 0.02 ± 0.02 | 3.88 ± 1.05 | 96.33 ± 3.67 | 99.49 ± 0.64 |
| 2-7 | 3 | − | 4.22 ± 0.06 | 3.37 ± 0.21 | 3.84 ± 0.30 | — | — |
| 2-8 | 3 | + | 0.06 ± 0.08 | 0.02 ± 0.02 | 4.50 ± 1.03 | 98.81 ± 2.07 | 99.60 ± 0.68 |
| 2-9 | 4 | − | 7.71 ± 0.10 | 6.35 ± 0.04 | 7.60 ± 0.05 | — | — |
| 2-10 | 4 | + | 0.03 ± 0.01 | 0.02 ± 0.00 | 9.32 ± 0.14 | 99.67 ± 0.58 | 100.00 ± 0.00 |
| 2-11 | 5 | − | 7.16 ± 0.17 | 6.10 ± 0.18 | 7.30 ± 0.15 | — | — |
| 2-12 | 5 | + | 0.08 ± 0.07 | 0.03 ± 0.01 | 9.26 ± 0.14 | 98.67 ± 0.58 | 99.67 ± 0.58 |
| 2-13 | 6 | − | 7.74 ± 0.08 | 6.19 ± 0.18 | 7.28 ± 0.20 | — | — |
| 2-14 | 6 | + | 0.27 ± 0.32 | 0.03 ± 0.00 | 9.25 ± 0.19 | 96.57 ± 4.22 | 99.15 ± 0.26 |

### Example 12

As Example 12, a selective recovery test of gold and palladium from 4 M aqua regia was conducted using the spray-dried sample.

### (Test Nos. 3-1 and 3-2)

First, 15 mg of the spray-dried sample was added to 1 ml of a 4 M aqua regia solution containing 5 ppm each of Au³⁺, Pd²⁺, and Cu²⁺, and stirred at room temperature for 30 minutes. Next, the aqua regia solution was centrifuged (12,000 rpm, 1 minute), and the metal concentration of the obtained supernatant was measured using ICP-MS (Test No. 3-2).

In addition, as a control test, the same test as Test No. 3-2 was conducted except that the spray-dried sample was not added, and the metal concentration of the supernatant was measured (Test No. 3-1). Next, the recovery rate in Test No. 3-2 was calculated from the difference between the metal concentrations of Test No. 3-1 and Test No. 3-2 measured above. The measured metal concentration and the recovery rate are shown in Table 13. The metal concentrations and the recovery rates shown in Table 13 are the mean value of three independent tests±SD (standard deviation).

TABLE 13

| Test | | Metal concentration of supernatant | | | Recovery rate (%) | |
|---|---|---|---|---|---|---|
| No. | Powder | Au³⁺ | Pd²⁺ | Cu²⁺ | Au³⁺ | Pd²⁺ |
| 3-1 | − | 4.93 ± 1.55 | 4.71 ± 1.44 | 4.49 ± 1.27 | — | — |
| 3-2 | + | 0.69 ± 0.44 | 0.04 ± 0.02 | 3.80 ± 0.98 | 87.00 ± 4.36 | 99.33 ± 0.58 |

### Example 13

As Example 13, an elution test of gold and palladium was conducted using the spray-dried sample by which gold and palladium were selectively recovered from a 4 M aqua regia solution.

### (Test Nos. 4-1 and 4-2)

First, 20 mg of the spray-dried sample was added to 1 ml of a 4 M aqua regia solution containing 0.5 ppm each of Au³⁺, Pd²⁺, and Cu²⁺, and stirred at room temperature for 30 minutes. Next, the aqua regia solution was centrifuged (12,000 rpm, 1 minute), and the metal concentration of the obtained supernatant was measured using ICP-MS (Test No. 4-2). The measured metal concentration of the supernatant is shown in Table 14.

In addition, as a control test, the same test as Test No. 4-2 was conducted except that the spray-dried sample was not added, and the metal concentration of the supernatant was measured (Test No. 4-1). Next, from the difference between the metal concentrations of Test No. 4-1 and Test No. 4-2 measured above, the metal concentration recovered by the spray-dried sample in Test No. 4-2 was calculated. The measured metal concentration of the supernatant and the metal concentration recovered by the spray-dried sample are shown in Table 14.

Next, a spray-dried sample fraction (precipitated fraction) recovered by centrifugation in Test No. 4-2 was dissolved in 1 ml of a 4 M aqua regia solution and was washed by performing centrifugation (12,000 rpm, 1 minute) on the aqua regia solution. Next, 1 ml of a 0.2 M NH₄Cl/2.8% NH₃ solution (pH 11) was added as a metal elution solution to the spray-dried sample fraction (precipitated fraction) obtained by centrifugation of the aqua regia solution, and stirred at room temperature for 30 minutes. The solution was again centrifuged (12,000 rpm, 1 minute), and the metal concentration of the obtained supernatant was measured using ICP-MS. The measured value is shown in Table 14 as the metal concentration of the eluate.

In addition, the elution rate in Test No. 4-2 was calculated using Formula (3). The calculated elution rate is shown in Table 14.

$$\begin{matrix}[ Formula 3 ] & \\ Elution rate ( \% ) = metal concentration of eluate / metal concentration recovered by spray - dried sample \times 100 & ( 3 )\end{matrix}$$

### (Test Nos. 4-3 to 4-10)

Test Nos. 4-3 to 4-10 were conducted in the same manner as in Test No. 4-1 or 4-2 except that the concentration of each metal contained in the 4 M aqua regia solution was changed to the concentration shown in Table 14. For each test, the metal concentration of the supernatant, the metal concentration recovered by the spray-dried sample, the metal concentration of the eluate, and the elution rate are shown in Table 14.

TABLE 14

| | Initial | | | | | Recovered | | | | | | | |
|---|---|---|---|---|---|---|---|---|---|---|---|---|---|
| | metal | | Metal | | | metal | | | Metal | | | | |
| | concen- | | concentration of | | | concentration | | | concentration of | | | Elution | |
| Test | tration | | supernatant (ppm) | | | (ppm) | | | eluate (ppm) | | | rate (%) | |
| No. | (ppm) | Powder | Au³⁺ | Pd²⁺ | Cu²⁺ | Au³⁺ | Pd²⁺ | Cu²⁺ | Au³⁺ | Pd²⁺ | Cu²⁺ | Au³⁺ | Pd²⁺ |
| 4-1 | 0.5 | − | 0.4 | 0.4 | 0.4 | — | — | — | — | — | — | — | — |
| 4-2 | 0.5 | + | 0.07 | 0.0 | 0.5 | 0.3 | 0.4 | 0.0 | 0.3 | 0.2 | 0.0 | 91 | 58 |
| 4-3 | 5 | − | 5.4 | 3.7 | 3.8 | — | — | — | — | — | — | — | — |
| 4-4 | 5 | + | 0.5 | 0.02 | 3.6 | 4.9 | 3.7 | 0.2 | 1.1 | 2.4 | 0.0 | 23 | 66 |
| 4-5 | 10 | − | 9.8 | 7.1 | 7.3 | — | — | — | — | — | — | — | — |
| 4-6 | 10 | + | 1.3 | 0.08 | 6.9 | 8.5 | 7.0 | 0.4 | 1.6 | 5.7 | 0.0 | 19 | 81 |
| 4-7 | 25 | − | 25 | 18 | 19 | — | — | — | — | — | — | — | — |
| 4-8 | 25 | + | 4.5 | 0.9 | 16 | 21 | 17 | 2.8 | 3.2 | 12 | 0.0 | 16 | 71 |
| 4-9 | 50 | − | 50 | 32 | 36 | — | — | — | — | — | — | — | — |
| 4-10 | 50 | + | 20 | 5.6 | 57 | 31 | 26 | 0.0 | 7.0 | 21 | 0.0 | 23 | 81 |

### Example 14

As Example 14, a recovery test of gold from a 0.1 mM KOH solution (pH 10) containing cyan and gold was conducted using the spray-dried sample. In the 0.1 mM KOH solution, Au³⁺ ions are stably present as a cyanide complex.

### (Test Nos. 5-1 and 5-2)

First, 15 mg of the spray-dried sample was added to 1 ml of 0.1 mM KOH (pH 10) containing 1 ppm of gold and stirred at room temperature for 30 minutes. Next, the KOH solution was centrifuged (12,000 rpm, 1 minute), and the metal concentration of the obtained supernatant was measured using ICP-MS (Test No. 5-2).

In addition, as a control test, the same test as Test No. 5-2 was conducted except that the spray-dried sample was not added, and the metal concentration of the supernatant was measured (Test No. 5-1). Next, the recovery rate in Test No. 5-2 was calculated from the difference between the metal concentrations of Test No. 5-1 and Test No. 5-2 measured above. The measured metal concentration and the recovery rate are shown in Table 15. The metal concentrations and the recovery rates shown in Table 15 are the mean value of two independent tests.

TABLE 15

| Test | | Au³⁺ concentration | Recovery |
|---|---|---|---|
| No. | Powder | of supernatant (ppm) | rate (%) |
| 5-1 | − | 1.0 | — |
| 5-2 | + | 0.23 | 77 |

### Example 15

As Example 15, a selective recovery test of platinum, gold, palladium, and osmium from a hydrochloric acid solution (1 to 4 M) was conducted using the spray-dried sample.

### (Test Nos. 6-1 and 6-2)

First, 10 mg of the spray-dried sample was added to 1 ml of a 1 M hydrochloric acid solution containing 10 ppm each of Rh³⁺, Pd²⁺, Os⁴⁺, Pt²⁺, Au³⁺, and Ir³⁺, and stirred overnight at room temperature. Next, the hydrochloric acid solution was centrifuged (12,000 rpm, 1 minute), and the metal concentration of the obtained supernatant was measured using ICP-MS (Test No. 6-2). In addition, as a control test, the same test as Test No. 6-2 was conducted except that the spray-dried sample was not added, and the metal concentration of the supernatant was measured (Test No. 6-1). The measured metal concentrations are shown in Table 16.

Next, the recovery rate in Test No. 6-2 was calculated from the difference between the metal concentrations of Test No. 6-1 and Test No. 6-2 measured above. The calculated recovery rate is shown in Table 17. The metal concentrations shown in Table 16 and the recovery rates shown in Table 17 are the mean value of three independent tests±SD (standard deviation).

### (Test Nos. 6-3 to 6-8)

Test Nos. 6-3 to 6-8 were conducted in the same manner as in Test No. 6-1 or 6-2 except that the concentration of the hydrochloric acid solution was changed to the concentration shown in Tables 16 and 17. For each test, the measured metal concentration is shown in Table 16, and the recovery rate is shown in Table 17.

TABLE 16

| | HCl | | | | | | | |
|---|---|---|---|---|---|---|---|---|
| Test | concentration | | Metal concentration of supernatant (ppm) | | | | | |
| No. | (mol/L | powder | Rh³⁺ | Pd²⁺ | Os⁴⁺ | Pt²⁺ | Au³⁺ | Ir³⁺ |
| 6-1 | 1 | − | 11.0 ± 1.8 | 10.0 ± 1.8 | 11.0 ± 1.6 | 10.0 ± 1.8 | 10.0 ± 1.9 | 10.0 ± 1.6 |
| 6-2 | 1 | + | 8.3 ± 0.1 | 0.0 ± 0.2 | 4.3 ± 0.1 | 0.6 ± 0.1 | 0.1 ± 0.1 | 6.7 ± 0.3 |
| 6-3 | 2 | − | 10.0 ± 1.4 | 10.0 ± 1.4 | 10.0 ± 1.3 | 10.0 ± 1.4 | 10.0 ± 1.4 | 10.0 ± 1.3 |
| 6-4 | 2 | + | 9.1 ± 0.1 | 0.0 ± 0.6 | 6.2 ± 0.5 | 4.5 ± 0.4 | 0.1 ± 0.1 | 8.7 ± 0.9 |
| 6-5 | 3 | − | 11 ± 1.7 | 11.0 ± 1.7 | 10.7 ± 1.5 | 11.0 ± 1.7 | 11.0 ± 1.7 | 11.0 ± 1.6 |
| 6-6 | 3 | + | 9.3 ± 0.1 | 0.0 ± 0.8 | 6.6 ± 0.6 | 5.9 ± 0.8 | 0.1 ± 0.1 | 9.1 ± 1.0 |
| 6-7 | 4 | − | 10.0 ± 1.2 | 10.0 ± 1.2 | 10.0 ± 1.1 | 10.0 ± 1.1 | 10.0 ± 1.2 | 10.0 ± 1.1 |
| 6-8 | 4 | + | 9.0 ± 0.1 | 0.0 ± 0.6 | 6.5 ± 0.4 | 6.1 ± 0.9 | 0.1 ± 0.1 | 8.7 ± 0.6 |

TABLE 17

| | HCl | | | | | | | |
|---|---|---|---|---|---|---|---|---|
| Test | concentration | | Recovery rate (%) | | | | | |
| No. | (mol/L) | powder | Rh³⁺ | Pd²⁺ | Os⁴⁺ | Pt²⁺ | Au³⁺ | Ir³⁺ |
| 6-2 | 1 | + | 19 ± 12 | 100 ± 0.6 | 59 ± 6.2 | 95 ± 1.4 | 99 ± 0.5 | 35 ± 8.3 |
| 6-4 | 2 | + | 12 ± 2.4 | 100 ± 0.6 | 41 ± 1.8 | 56 ± 2.1 | 99 ± 0.7 | 16 ± 1.6 |
| 6-6 | 3 | + | 14 ± 8.7 | 100 ± 0.5 | 38 ± 6.1 | 46 ± 4.3 | 100 ± 0.7 | 16 ± 8.4 |
| 6-8 | 4 | + | 12 ± 6.2 | 100 ± 0.5 | 35 ± 4.8 | 39 ± 7.3 | 99 ± 0.8 | 13 ± 6.0 |

### Example 16

As Example 16, a selective recovery test of ruthenium from a hydrochloric acid solution (1 or 2 M) was conducted using the spray-dried sample.

### (Test Nos. 7-1 and 7-2)

First, 20 mg of the spray-dried sample was added to 1 ml of a 1 M hydrochloric acid solution containing 10 ppm of Ru³⁺ and stirred overnight at room temperature. Next, the hydrochloric add solution was centrifuged (12,000 rpm, 1 minute), and the metal concentration of the obtained supernatant was measured using ICP-MS (Test No. 7-2). In addition, as a control test, the same test as Test No. 7-2 was conducted except that the spray-dried sample was not added, and the metal concentration of the supernatant was measured (Test No. 7-1). The measured metal concentration is shown in Table 18.

Next, the recovery rate in Test No. 7-2 was calculated from the difference between the metal concentrations of Test No. 7-1 and Test No. 7-2 measured above. The calculated recovery rate is shown in Table 18. The metal concentrations and the recovery rates shown in Table 18 are the mean value of three independent tests±SD (standard deviation).

### (Test Nos. 7-3 to 7-4)

Test Nos. 7-3 and 7-4 were conducted in the same manner as in Test No. 7-1 or 7-2 except that the concentration of the hydrochloric acid solution was changed to the concentration shown Table 18. For each test, the measured metal concentration and the recovery rate are shown in Table 18.

TABLE 18

| | HCl | | Ru³⁺ concentration | Ru³⁺ |
|---|---|---|---|---|
| Test | concentration | | of supernatant | recovery rate |
| No. | (mol/L) | powder | (ppm) | (%) |
| 7-1 | 1 | − | 10.3 ± 0.2 | — |
| 7-2 | 1 | + | 5.6 ± 0.2 | 45.6 ± 2.3 |
| 7-3 | 2 | − | 10.5 ± 0.2 | — |
| 7-4 | 2 | + | 5.5 ± 0.3 | 47.9 ± 2.6 |

### Comparative Example 1

As Comparative Example 1, a selective recovery test of metal from 0.43 M aqua regia was conducted using live cells of G. sulphuraria.

(Test Nos. C1-1 and C1-2)

First, 14 mg (dry weight) of live cells of G. sulphuraria were added to 1 ml of a 0.43 M aqua regia solution containing 871 ppm of Au³⁺, 86 ppm of Fe²⁺/³⁺, 107 ppm of Cu²⁺, 4.5 ppm of Pt⁴⁺, 262 ppm of Ni²⁺, and 24 ppm of Sn²⁺, and stirred at room temperature for 30 minutes. Next, the aqua regia solution was centrifuged (12,000 rpm, 1 minute), and the metal concentration of the obtained supernatant was measured using ICP-MS (Test No. C1-2).

In addition, as a control test, the same test as Test No. C1-2 was conducted except that live cells of G. sulphuraria were not added, and the metal concentration of the supernatant was measured (Test No. C1-1). Next, the recovery rate in Test No. C1-2 was calculated from the difference between the metal concentrations of Test No. C1-1 and Test No. C1-2 measured above. The measured metal concentration and the recovery rate are shown in Table 19. The metal concentrations and the recovery rates shown in Table 19 are the mean value of three independent tests±SD (standard deviation).

TABLE 19

| | | Metal concentration of supernatant | | | | | | | | | | | |
|---|---|---|---|---|---|---|---|---|---|---|---|---|---|
| | | (ppm) | | | | | | Recovery rate (%) | | | | | |
| Test | | Fe²⁺/ | | | | | | Fe²⁺/ | | | | | |
| No. | Cells | Fe³⁺ | Cu²⁺ | Pt⁴⁺ | Au³⁺ | Ni²⁺ | Sn²⁺ | Fe³⁺ | Cu²⁺ | Pt⁴⁺ | Au³⁺ | Ni²⁺ | Sn²⁺ |
| C1-1 | − | 86 ± 14 | 107 ± 44 | 4.5 ± 0.6 | 871 ± 86 | 262 ± 48 | 24 ± 3.5 | — | — | — | — | — | — |
| C1-2 | + | 63 ± 7.6 | 73 ± 11 | 3.7 ± 0.3 | 299 ± 108 | 223 ± 19 | 18 ± 1.3 | 26 | 28 | 17 | 66 | 14 | 23 |

### Comparative Example 2

As Comparative Example 2, a selective recovery test of metal from 4 M aqua regia was conducted using live cells of G. sulphuraria.

(Test Nos. C2-1 and C2-2)

First, 14 mg (dry weight) of live cells of G. sulphuraria were added to 1 ml of a 4 M aqua regia solution containing 87 ppm of Fe²⁺/³⁺, 56 ppm of Cu²⁺, 9 ppm of Sn²⁺, 3 ppm of Pt⁴⁺, and 912 ppm of Au³⁺, and stirred at room temperature for 30 minutes. Next, the aqua regia solution was centrifuged (12,000 rpm, 1 minute), and the metal concentration of the obtained supernatant was measured using ICP-MS (Test No. C2-2).

In addition, as a control test, the same test as Test No. C2-2 was conducted except that live cells of G. sulphuraria were not added, and the metal concentration of the supernatant was measured (Test No. C2-1). Next, the recovery rate in Test No. C2-2 was calculated from the difference between the metal concentrations of Test No. C2-1 and Test No. C2-2 measured above. The measured metal concentration and the recovery rate are shown in Table 20.

TABLE 20

| | | Metal concentration of supernatant (ppm) | | | | | Recovery rate (%) | | | | |
|---|---|---|---|---|---|---|---|---|---|---|---|
| Test | | Fe²⁺/ | | | | | Fe²⁺/ | | | | |
| No. | Cells | Fe³⁺ | Cu²⁺ | Sn²⁺ | Pt⁴⁺ | Au³⁺ | Fe³⁺ | Cu²⁺ | Sn²⁺ | Pt⁴⁺ | Au³⁺ |
| C2-1 | − | 87 | 56 | 9 | 3 | 912 | — | — | — | — | — |
| C2-2 | + | 120 | 60 | 10 | 4 | 794 | 0 | 0 | 0 | 0 | 13 |

### Example 17

As Example 17, an elution test of gold was conducted using the spray-dried sample by which gold was recovered from a 4 M aqua regia solution and an ion exchange resin.

### (Test No. 8-1)

First, 20 mg of the spray-dried sample was added to 1 ml of a 4 M aqua regia solution containing 10 ppm of Au³⁺, and stirred at room temperature for 30 minutes. Next, the aqua regia solution was centrifuged (12,000 rpm, 1 minute), and the metal concentration of the obtained supernatant was measured using ICP-MS (Test No. 8-1). In addition, the same test as Test No. 8-1 was conducted except that spray-dried sample was not added, and the metal concentration of the supernatant was measured (Test No. 8-1-control). Next, from the difference between the metal concentrations of Test No. 8-1 and Test No. 8-1-control measured above, the metal concentration recovered by the spray-dried sample in Test No. 8-1 was calculated.

Next, a spray-dried sample fraction (precipitated fraction) recovered by centrifugation in Test No. 8-1 was dissolved in 1 ml of a 4 M aqua regia solution and was washed by performing centrifugation (12,000 rpm, 1 minute) on the aqua regia solution. Next, 1 ml of 4 M HCl was added as a metal elution solution to the spray-dried sample fraction (precipitated fraction) obtained by centrifugation of the aqua regia solution, and stirred at room temperature for 30 minutes. The solution was again centrifuged (12,000 rpm, 1 minute), and the metal concentration of the obtained supernatant was measured using ICP-MS. The measured value was used as the metal concentration of the eluate.

Next, the elution rate in Test No. 8-1 was calculated using Formula (3). The calculated elution rate is shown in Table 21. The elution rates of Test Nos. 8-1 to 8-5 shown in Table 21 are the mean value of three independent tests±SD (standard deviation).

### (Test Nos. 8-2 to 8-5)

Test Nos. 8-2 to 8-5 were conducted in the same manner as in Test No. 8-1 except that as the metal elution solution, instead of 4 M HCl, 0.5 M EDTA (pH 8) (Test No. 8-2), 1 M Thiourea/0.1 M HCl (Test No. 8-3), 1 M KOH (Test No. 8-4), and 6 M aqua regia (Test No. 8-5) were used. For each test, the elution rate is shown in Table 21.

### (Test Nos. 8-6 to 8-10)

Test Nos. 8-6 to 8-10 were conducted in the same manner as in Test No. 8-1 except that an ion exchange resin (SA10A, manufactured by Mitsubishi Chemical Corporation) was used as the metal recovery agent instead of the spray-dried sample, and one those shown in Table 21 were used as the metal elution solutions. For each test, the elution rate is shown in Table 21.

TABLE 21

| Test | | Metal recovery | Recovery |
|---|---|---|---|
| No. | Metal elution solution | agent | rate (%) |
| 8-1 | 4M HCl | Spray-dried sample | 25 ± 2.2 |
| 8-2 | 0.5M EDTA (pH 8) | | 20 ± 6.4 |
| 8-3 | 1M Thiourea/0.1M HCl | | 93 ± 5.7 |
| 8-4 | 1M KOH | | 67 ± 5.3 |
| 8-5 | 6M aqua regia | | 60 ± 12 |
| 8-6 | 4M HCl | Ion exchange resin | 0 |
| 8-7 | 0.5M EDTA (pH 8) | | 1.1 |
| 8-8 | 1M Thiourea/0.1M HCl | | 72 |
| 8-9 | 1M KOH | | 12 |
| 8-10 | 6M aqua regia | | 5.4 |

### Example 18

As Example 18, an elution test of palladium was conducted using the freeze-dried sample by which palladium was selectively recovered from a 5 M aqua regia solution.

### (Test Nos. 9-1 and 9-2)

First, 120 mg of the freeze-dried sample was added to 30 ml of a 5 M aqua regia solution containing 10 ppm each of Pd²⁺, Al³⁺, Cd²⁺, Fe²⁺/³⁺, Mn²⁺, Pb²⁺, and Zn²⁺, and stirred at room temperature for 30 minutes. Next, the aqua regia solution was divided into 10 ml (Test No. 9-1, concentration ratio 10 times) and 20 ml (Test No. 9-2, concentration ratio 20 times) and centrifuged (12,000 rpm, 1 minute), and the metal concentration of each of the obtained supernatants was measured using ICP-MS (Test Nos. 9-1 and 9-2). In addition, the same test as Test Nos. 9-1 and 9-2 was conducted except that the freeze-dried sample was not added, and the metal concentration of the supernatant was measured (Test Nos 9-1-control and 9-2-control). Next, from the difference between the metal concentrations of Test No. 9-1 and Test No. 9-1-control measured above, the metal concentration recovered by the freeze-dried sample in Test No. 9-1 was calculated. Similarly, from the difference between the metal concentrations of Test No. 9-2 and Test No. 9-2-control measured above, the metal concentration recovered by the freeze-dried sample in Test No. 9-2 was calculated.

Next, freeze-dried sample fractions (precipitated fractions) recovered by centrifugation in Test Nos. 9-1 and 9-2 were each dissolved in 1 ml of a 5 M aqua regia solution and were washed by performing centrifugation (12,000 rpm, 1 minute) on the aqua regia solution. Next, 1 ml of 6 M aqua regia was added as a metal elution solution to each of the freeze-dried sample fractions (precipitated fractions) obtained by centrifugation of the aqua regia solution, and stirred overnight at room temperature. The solution was again centrifuged (12,000 rpm, 1 minute), and the metal concentration of each of the obtained supernatants was measured using ICP-MS. The measured values are shown in Table 22 as the metal concentrations of the eluates.

Next, the elution rates in Test Nos. 9-1 and 9-2 were calculated using Formula (3). The calculated elution rates are shown in Table 23.

TABLE 22

| | | Metal concentration of eluate (ppm) | | | | | | |
|---|---|---|---|---|---|---|---|---|
| Test | Concentration | | | | Fe²⁺/ | | | |
| No. | ratio | Pd²⁺ | Al³⁺ | Cd²⁺ | Fe³⁺ | Mn²⁺ | Pb²⁺ | Zn²⁺ |
| 9-1 | 10 times | 51 | 2.9 | 0.3 | 0 | 0.2 | 0 | 0.8 |
| 9-2 | 20 times | 87 | 1.8 | 0.2 | 0.1 | 0.2 | 0 | 0.6 |

TABLE 23

| Test No. | Concentration ratio | Elution rate (%) |
|---|---|---|
| 9-1 | 10 times | 99 |
| 9-2 | 20 times | 85 |

### Example 19

As Example 19, a selective recovery test of iridium from a hydrochloric acid solution (0.1 to 1 M) was conducted using the spray-dried sample.

### (Test No. 10-1)

First, 20 mg of the spray-dried sample was added to 1 ml of a 0.1 M hydrochloric acid solution containing 10 ppm of Ir³⁺, and stirred overnight at room temperature. Next, the hydrochloric acid solution was centrifuged (12,000 rpm, 1 minute), and the metal concentration of the obtained supernatant was measured using ICP-MS (Test No. 10-1). In addition, as a control test, the same test as Test No. 10-1 was conducted except that the spray-dried sample was not added, and the metal concentration of the supernatant was measured (Test No. 10-1-control). Next, the recovery rate in Test No. 10-1 was calculated from the difference between the metal concentrations of Test No. 10-1 and Test No. 10-1-control measured above. The calculated recovery rate is shown in Table 24. The recovery rate shown in Table 24 is the mean value of three independent tests±SD (standard deviation).

### (Test Nos. 10-2 to 10-4)

Test Nos. 10-2 to 10-4 were conducted in the same manner as in Test No. 10-1 except that the concentration of the hydrochloric acid solution was changed to the concentration shown Table 24. For each test, the calculated recovery rate is shown in Table 24.

TABLE 24

| Test No. | HCl concentration (mol/l) | Recovery rate (%) |
|---|---|---|
| 10-1 | 0.1 | 87.0 ± 0.90 |
| 10-2 | 0.2 | 78.9 ± 0.72 |
| 10-3 | 0.5 | 55.3 ± 0.31 |
| 10-4 | 1 | 31.9 ± 6.40 |

### INDUSTRIAL APPLICABILITY

According to the present invention, an agent for selective metal recovery, a metal recovery method, and a metal elution method, by which selective recovery, elution, purification and the like of metals can be efficiently carried out at low cost, can be provided. Therefore, the present invention is highly valuable for industrial utilization in recycling of noble metals or rare metals, such as the separation of rare earth elements from metal effluent containing iron and reduction and recovery of gold ions, recovery of noble metals or rare metals included at low concentrations in the environment, elution or purification of noble metal ion complexes from living organisms or adsorbent materials, and the like.

### Embodiments

Embodiment 1. An agent for selective metal recovery, the agent comprising a material derived from an alga belonging to the order Cyanidiales, which is dead cells or a cell surface layer of an alga belonging to the order Cyanidiales, or an artificial material produced by simulating the cell surface layer, or comprising a porphyrin.

Embodiment 2. The agent for selective metal recovery according to embodiment 1, wherein the porphyrin is coproporphyrin and/or pheophytin.

Embodiment 3. The agent for selective metal recovery according to embodiment 1 or 2, wherein the porphyrin is a protonated compound.

Embodiment 4. The agent for selective metal recovery according to any one of embodiments 1 to 3, wherein the agent selectively recovers a noble metal and/or a rare metal including a rare earth element.

Embodiment 5. The agent for selective metal recovery according to any one of embodiments 1 to 4, wherein the agent selectively recovers a noble metal including gold or palladium, and/or a lanthanoid from a base metal mixture solution under acidic conditions.

Embodiment 6. The agent for selective metal recovery according to embodiment 5, wherein the agent separates and selectively recovers a lanthanoid and iron based on the difference between the ionic radii of the respective elements and the degree of stability of complexes.

Embodiment 7. The agent for selective metal recovery according to embodiment 1, wherein the cell surface layer of an alga belonging to the order Cyanidiales adsorbs a noble metal ion complex by an electrostatic interaction or ion exchange and desorbs the noble metal ion complex with a predetermined solution.

Embodiment 8. The agent for selective metal recovery according to any one of embodiments 1 to 7, wherein the porphyrin forms nanoparticles by reducing a noble metal.

Embodiment 9. A metal recovery method, comprising:
an addition step of adding a material derived from an alga belonging to the order Cyanidiales, which is dead cells or a cell surface layer of an alga belonging to the order Cyanidiales, or an artificial material produced by simulating the cell surface layer, or adding a porphyrin, to a metal solution; and
a recovery step of recovering a metal from the metal solution by the material derived from an alga belonging to the order Cyanidiales or the porphyrin.

Embodiment 10. The metal recovery method according to embodiment 9, wherein the porphyrin is coproporphyrin and/or pheophytin.

Embodiment 11. The metal recovery method according to embodiment 9 or 10, wherein the recovery step is a step of selectively recovering a noble metal and/or a rare metal including a rare earth element from the metal solution.

Embodiment 12. The metal recovery method according to any one of embodiments 9 to 11, wherein the recovery step involves selective recovery of a noble metal including gold or palladium, and/or a lanthanoid from a base metal mixture solution under acidic conditions.

Embodiment 13. The metal recovery method according to embodiment 12, wherein the recovery step involves separation and selective recovery of a lanthanoid and iron based on the difference between the ionic radii of the respective elements and the degree of stability of complexes.

Embodiment 14. The metal recovery method according to anyone of embodiments 9 to 13, further comprising a reduction step of forming nanoparticles by causing the porphyrin to reduce a noble metal.

Embodiment 15. The metal recovery method according to any one of embodiments 9 to 14, wherein the recovery step involves recovery of gold ions by adsorption using the material derived from an alga belonging to the order Cyanidiales, and the method comprises a step of reducing gold ions by a reducing action of the porphyrin.

Embodiment 16. A metal elution method for eluting a noble metal including gold or palladium, which has been recovered into a material derived from an alga belonging to the order Cyanidiales, which is dead cells or a cell surface layer of an alga belonging to the order Cyanidiales, or an artificial material produced by simulating the cell surface layer,
the method comprising a step of adding a composition for metal elution, which is an acidic solution, to the material derived from an alga belonging to the order Cyanidiales.

Embodiment 17. A metal elution method for eluting a metal which has been recovered into a material derived from an alga belonging to the order Cyanidiales, which is dead cells or a cell surface layer of an alga belonging to the order Cyanidiales, or an artificial material produced by simulating the cell surface layer,
the method comprising a step of adding a composition for metal elution including a mixed liquid of ammonia and an ammonium salt to the material derived from an alga belonging to the order Cyanidiales.

Embodiment 18. A metal recovery agent or a metal compound recovery agent comprising:
a dried matter of a cell of red algae belonging to the order Cyanidiales;
a dried matter of a cell derivative of red algae belonging to the order Cyanidiales; or
an artificial matter that imitates the dried matter of the cell or the dried matter of the cell derivative.

Embodiment 19. A method for recovering a metal or a metal compound, comprising:
an addition step of adding a dried matter of a cell of the red algae belonging to the order Cyanidiales, a dried matter of a cell derivative of the red algae belonging to the order Cyanidiales, or an artificial matter that imitates the dried matter of the cells or the dried matter of the cell derivative to a metal solution; and
an adsorption step of causing a metal or a metal compound contained in the metal solution to be adsorbed onto the cell derived from the dried matter, the cell derivative derived from the dried matter, or the artificial matter.

Embodiment 20. The method for recovering a metal or a metal compound according to embodiment 19,
wherein an amount of the dried matter or the artificial matter added in the addition step is 0.001 mg or more with respect to 100 ml of the metal solution.

Embodiment 21. The method for recovering a metal or a metal compound according to embodiment 19 or 20,
wherein the adsorption step is a step of causing at least one metal selected from the group consisting of gold, palladium, ruthenium, platinum, iridium, and osmium, or at least one metal compound containing the metal selected from the group consisting of gold, palladium, ruthenium, platinum, iridium, and osmium contained in the metal solution to be selectively adsorbed.

Embodiment 22. The method for recovering a metal or a metal compound according to anyone of embodiments 19 to 21,
wherein an acid concentration of the metal solution is 0.5 mmol/L or more.

Embodiment 23. The method for recovering a metal or a metal compound according to embodiment 19 or 20,
wherein the adsorption step is a step of recovering a gold cyanide complex contained in the metal solution.

Embodiment 24. The method for recovering a metal or a metal compound according to anyone of embodiments 19 to 23, further comprising:
a step of refining the metal or the metal compound adsorbed onto the cell derived from the dried matter, the cell derivative derived from the dried matter, or the artificial matter.

Embodiment 25. The method for recovering a metal or a metal compound according to anyone of embodiments 19 to 23, further comprising:
an elution step of eluting the metal or the metal compound adsorbed onto the cell derived from the dried matter, the cell derivative derived from the dried matter, or the artificial matter using a mixed solution containing ammonia and an ammonium salt.

1. A method for recovering a metal or a metal compound, comprising:
an addition step of adding a dried matter of a cell of the red algae belonging to the order Cyanidiales, a dried matter of a cell derivative of the red algae belonging to the order Cyanidiales, or an artificial matter that imitates the dried matter of the cells or the dried matter of the cell derivative to a metal solution; and
an adsorption step of causing a metal or a metal compound contained in the metal solution to be adsorbed onto the cell derived from the dried matter, the cell derivative derived from the dried matter, or the artificial matter.

2. The method for recovering a metal or a metal compound according to claim 1,
wherein an amount of the dried matter or the artificial matter added in the addition step is 0.001 mg or more with respect to 100 ml of the metal solution.

3. The method for recovering a metal or a metal compound according to claim 1,
wherein the adsorption step is a step of causing at least one metal selected from the group consisting of gold, palladium, ruthenium, platinum, iridium, and osmium, or at least one metal compound containing the metal selected from the group consisting of gold, palladium, ruthenium, platinum, iridium, and osmium contained in the metal solution to be selectively adsorbed.

4. The method for recovering a metal or a metal compound according to claim 1,
wherein an acid concentration of the metal solution is 0.5 mmol/L or more.

5. The method for recovering a metal or a metal compound according to claim 1,
wherein the adsorption step is a step of recovering a gold cyanide complex contained in the metal solution.

6. The method for recovering a metal or a metal compound according to claim 1 further comprising:
a step of refining the metal or the metal compound adsorbed onto the cell derived from the dried matter, the cell derivative derived from the dried matter, or the artificial matter.

7. The method for recovering a metal or a metal compound according to claim 1, further comprising:
an elution step of eluting the metal or the metal compound adsorbed onto the cell derived from the dried matter, the cell derivative derived from the dried matter, or the artificial matter using a mixed solution containing ammonia and an ammonium salt.

8. A metal recovery method, comprising:
an addition step of adding a material derived from an alga belonging to the order Cyanidiales, which is dead cells or a cell surface layer of an alga belonging to the order Cyanidiales, or an artificial material produced by simulating the cell surface layer; and
a recovery step of recovering a metal from the metal solution by the material derived from an alga belonging to the order Cyanidiales.

9. The metal recovery method according to claim 8, wherein the recovery step is a step of selectively recovering a noble metal and/or a rare metal including a rare earth element from the metal solution.

10. The metal recovery method according to claim 8, wherein the recovery step involves selective recovery of a noble metal including gold or palladium, and/or a lanthanoid from a base metal mixture solution under acidic conditions.

11. The metal recovery method according to claim 10, wherein the recovery step involves separation and selective recovery of a lanthanoid and iron based on the difference between the ionic radii of the respective elements and the degree of stability of complexes.

12. The metal recovery method according to claim 11, wherein the recovery step involves recovery of gold ions by adsorption using the material derived from an alga belonging to the order Cyanidiales.
